(19) United States
(12) Patent Application Publication
(10) Pub. No.: US 2023/0068487 A1
(43) Pub. Date: Mar. 2, 2023

(54) ALPHA-1-ANTITRYPSIN MUTANTS, COMPOSITIONS COMPRISING SAME, AND USE THEREOF

(71) Applicants: B. G. NEGEV TECHNOLOGIES AND APPLICATIONS LTD., AT BEN-GURION UNIVERSITY, Beer Sheva (IL); NATIONAL INSTITUTE FOR BIOTECHNOLOGY IN THE NEGEV LTD., Beer-Sheva (IL)

(72) Inventors: Yotam LIOR, Beer Sheva (IL); Eli Chaim LEWIS, Beer Sheva (IL)

(21) Appl. No.: 17/795,987

(22) PCT Filed: Jan. 28, 2021
(86) PCT No.: PCT/IL2021/050099

§ 371 Date: Jul. 28, 2022

Related U.S. Application Data
(60) Provisional application No. 62/966,629, filed on Jan. 28, 2020.

(51) Int. Cl. C07K 14/81; A61P 29/00

(52) U.S. Cl. CPC C07K 14/8125; A61P 29/00; A61K 38/00

(57) ABSTRACT

The present invention includes mutants of human alpha-1-antitrypsin (mhAAT). Further provided are compositions including the mhAAT and use of same, such as for modulating an immune cell, and treating a condition, such as inflammation.

### CROSS-REFERENCE TO RELATED APPLICATIONS

This application claims the benefit of priory of U.S. Provisional Patent Application No. 62/966,629 titled: “AL PHA-1-ANTITRYPSIN MUTANTS, COMPOSITIONS COMPRISING SAME, AND USE THEREOF” filed Jan. 28, 2020, the contents of which are incorporated herein by reference in their entirety.

### FIELD OF INVENTION

The present invention, in some embodiments, is directed to mutated forms of alpha 1-antitrypsin, compositions comprising same, and use thereof.

### BACKGROUND

Human al-antitrypsin (hAAT) is a 52 kDa, 394 amino acid long, evolutionary conserved glycoprotein. While hAAT serum levels are maintained mainly by hepatocytes, local secretion to a minor extent has been documented in lung epithelia, macrophages and intestinal epithelial cells. The promotor to hAAT gene is known to react to hypoxia, interleukin (IL)-1, IL-6 and cortisol, allowing up to a 6-fold increase of serum hAAT levels under stress conditions, defining the protein as an acute phase reactant.

hAAT is a member of the serine protease inhibitors (SERPINs) superfamily, utilizing a short 8 amino acid protruding segment (Reactive Center Loop, RCL, positions 357-366) as a specific bait for serine proteases. Based on the sequence of its RCL, hAAT inhibits the activity of a specific range of serine proteases with highest affinity to neutrophil elastase (NE). Other serine proteases were also shown to be inhibited by hAAT including chymotrypsin, cathepsin G, trypsin, plasmin and thrombin and even metalloproteases such as MMP9 or ADAMS17, albeit to a lower extent. The classical inhibition of a serine protease by AAT involves the binding of the protease to the RCL, which triggers a series of events resulting in the cleavage of the peptide bond between Met358 and Ser359, the formation of a covalent bond between hAAT and the attacking protease and conformational changes within hAAT exposing previously hidden segments. The new hAAT-protease complex is then removed from serum by hepatocytes.

Clinically, hAAT is mostly mentioned in association with a single gene disease called α1-antitrypsin deficiency (AATD). AATD is classically associated early-onset non-smokers lung emphysema and liver cirrhosis. However, recent data suggest an increased risk to other comorbidities to AATD, such as vasculitis, bacterial pneumonia, cervical artery dissection, type II diabetes mellitus, HIV infection, glomerulonephritis, poor wound healing, and even mood disorders. Currently, the sole clinically approved therapy for AATD is a life-long serum-purified hAAT weekly infusions, shown to be affective in postponing the pulmonary expression of the disease.

For many years it was assumed that protease—anti-protease imbalance was the key factor for the hallmark presentation of AATD: non-smokers pulmonary emphysema. However, given many yet-to-be-successful attempts to treat AATD with synthetic anti-protease compositions, this paradigm has started to shift in favor of a more elaborated and immune-related explanation.

Indeed, the anti-inflammatory and immunomodulatory repertoire of hAAT is extensive. Among these activities are the downregulation of pro-inflammatory cytokine levels, such as IL-6 and TNFα and upregulation of inflammation-driven anti-inflammatory agents such as IL-10 and IL-1Ra. hAAT was also shown to act as a scavenger and binding agent for various inflammation-associated agents such as reactive oxygen species (ROS), IL-8, and even danger molecules (DAMPs) such as heat shock protein (HSP)70 and glycoprotein (gp)96. Furthermore, hAAT exposure was shown to be associated with a more tolerogenic activation profile of dendritic cells and B lymphocyte, the expansion of antigen-specific regulatory T-cells and the induction of immune tolerance.

Interestingly, unlike many other anti-inflammatory drugs, which reduce the potency of immune reactions against wanted and unwanted targets alike, hAAT allows and even potentiates immune activity against genuine threats such as bacteria, and viruses, while minimizing tissue collateral damage.

While the biochemical mechanisms of hAAT protease-inhibiting activity were thoroughly described, its immune-associated functions remain poorly understood.

While being potentially applicable to a wide breadth of clinical conditions, concerns of the risks associated with infusion of human blood products as well as high production costs has limited hAAT use to conditions other than AATD. This dis-concordance has rendered hAAT as an attractive target for recombinant production, thus reducing production costs and improving safety. However, while many attempted to achieve this goal, clinical applications are yet to be approved.

In the past years, directed evolution methodologies have gained interest as both a potential way for study and generation of proteins with improved desirable activities. Examples of the application of these methodologies range from enhancement of thermostability and alteration of recombinant expression systems to improve enzymatic catalytic activity and alteration of substrate specificity.

Directed evolution methodologies are all based on the principles of natural selection and consist of two major steps: (1) generating gene libraries of the original gene after random or directed mutagenesis; and (2) selecting variants from the libraries based on desirable attributes.

There is still a great need for an hAAT variant having both improved anti-inflammatory and/or immunomodulatory functions, and protease-inhibiting capabilities.

### SUMMARY

According to a first aspect, there is provided a polypeptide comprising the amino acid sequence of SEQ ID NO: 9, wherein the polypeptide comprises at least one amino acid substitution at a position selected from the group consisting of: T22, T68, D74, D202, K243, and K368.

According to another aspect, there is provided an isolated polynucleotide molecule comprising a nucleic acid sequence encoding the polypeptide of the invention.

According to another aspect, there is provided an artificial vector comprising the isolated polynucleotide molecule of the invention.

According to another aspect, there is provided a cell comprising: (a) the polypeptide of the invention; (b) the isolated polynucleotide molecule the invention; (c) the artificial vector the invention; or (d) any combination of (a) to (c).

According to another aspect, there is provided a composition comprising: (a) the polypeptide of the invention; (b) the isolated polynucleotide molecule the invention; (c) the artificial vector the invention; (d) the cell of the invention; or (e) any combination of (a) to (d); and an acceptable carrier.

According to another aspect, there is provided a pharmaceutical composition comprising the polypeptide of the invention and a pharmaceutically acceptable carrier.

According to another aspect, there is provided a method for treating a subject afflicted with a condition selected from the group consisting of: AAT deficiency, an AAT related disease, and inflammation, comprising administering to the subject a therapeutically effective amount of any one of: (i) the polypeptide the invention; and (ii) the pharmaceutical composition the invention.

According to another aspect, there is provided a method for modulating the activity of an immune cell, comprising contacting the immune cell with an effective amount of the polypeptide the invention, thereby modulating the activity of the immune cell.

According to some embodiments, there is provided a method for producing a polypeptide characterized by having immune cell modulating activity, comprising: (a) providing a cell comprising the artificial vector of the invention; and (b) culturing the cell of step (a) such that a polypeptide encoded by the artificial vector is expressed, thereby producing the polypeptide characterized by having immune cell modulating activity.

In some embodiments, the polypeptide comprises the amino acid sequence of SEQ ID NO: 1, wherein X₁ is T or A; X₂ is T or A; X₃ is D or T; X₄ is D or H; X₅ is K or D; and X₆ is K or R.

In some embodiments, the polypeptide comprises the amino acid sequence of SEQ ID NO: 2.

In some embodiments, the polypeptide comprises the amino acid sequence of SEQ ID NO: 3.

In some embodiments, the polypeptide comprises the amino acid sequence of SEQ ID NO: 4.

In some embodiments, the polypeptide comprises the amino acid sequence of SEQ ID NO: 5.

In some embodiments, the polypeptide comprises the amino acid sequence of SEQ ID NO: 6.

In some embodiments, the polypeptide comprises the amino acid sequence of SEQ ID NO: 7.

In some embodiments, the polypeptide comprises the amino acid sequence of SEQ ID NO: 8.

In some embodiments, the artificial vector is an expression vector.

In some embodiments, the pharmaceutical composition is used in the treatment or prevention of a condition selected from the group consisting of: alpha antitrypsin (AAT) deficiency, an AAT related disease, and inflammation, in a subject in need thereof.

In some embodiments, any one of the polypeptide and the pharmaceutical composition reduce the abundance of a CD40^{Hi} cell, a CD86^{Hi} cell, or both, in the subject.

In some embodiments, any one of the polypeptide and the pharmaceutical composition reduce the expression level, the secretion level, or both, of a factor selected from the group consisting of: interleukin (IL)-6, and tumor necrosis factor alpha, in the subject.

In some embodiments, any one of the polypeptide and the pharmaceutical composition increase the expression level, the secretion level, or both, of a factor selected from the group consisting of: IL-10, and IL-1Ra, in the subject.

In some embodiments, the immune cell is a neutrophil or a macrophage.

In some embodiments, the activity comprises: anti-inflammatory activity, protease inhibiting activity, pro-inflammatory activity, or any combination thereof.

In some embodiments, the immune cell is a cell of a subject.

In some embodiments, the subject is afflicted with a condition selected from the group consisting of: AAT deficiency, an AAT related disease, inflammation, and any combination thereof.

In some embodiments, the contacting is contacting in vivo or in vitro.

In some embodiments, the polypeptide is the polypeptide of the invention.

In some embodiments, the method further comprises a step preceding step (a) comprising introducing or transfecting the cell with the artificial vector.

In some embodiments, the method further comprises a step proceeding step (b) comprising isolating, extracting, purifying, or any combination thereof, the polypeptide from the cell, from a medium wherein the cell is cultured, or both.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIGS. 1A-1B include computerized 3D model structures of human al Antitrypsin (hAAT) rendered from 1ATU.pdb file using VMD software. Dark gray, identified evolutionary flexible sites.

FIG. 2 includes a vertical bar graph showing the expression of recombinant hAAT (rhAAT) variants. Supernatant were collected from HEK-T293T transfected cells. Selected variants out of 103 variants were analyzed. Accumulated rhAAT values are represented as mean±standard error of the mean (SEM). Shown are representative results out of 2 independent experiments.

FIGS. 3A-3C include vertical bar graphs showing the anti-inflammatory properties of rhAAT variants in vitro. BMDM cells (3×10⁵ per well in a round bottom 24-well plate at a final volume of 300 μl, in triplicates) were introduced with 60 μl of rhAAT-transfected-HEK-T293F supernatants, incubated overnight, washed, and then stimulated by incubation with lipopolysaccharides (LPS; 5 ng/ml) for another 24 hours. (3A) Supernatant LPS-induced interleukin (IL)-6 levels were quantified by specific enzyme-linked immunosorbent assay (ELISA). (3B) Flow cytometric analyses were performed to quantify the membranal activation markers, CD40^{Hi} (3B) and CD86^{Hi} (3C). Gate, CD11b⁺/F4/80⁺ cells. Results are presented as % of positive control (non-rhAAT treated LPS-stimulated cells). Mean±SEM. * p<0.05, ** p<0.01, *** p<0.001. Representative results out of 3 independent experiments.

FIGS. 4A-4D include a ribbon diagram, an amino acid sequence, and micrographs of post-protein transfer nitrocellulose membrane analyses. (4A) Computerized 3-dimensional (3D) model of hAAT rendered from 1ATU.pdb file using VMD. The following mutation sites and substitutions are indicated by arrows: Threonine 22 to Alanine (T22A); Threonine 68 to Alanine (T68A); Aspartic acid to Threonine (D74T); Aspartic acid 202 to Histidine (D202H); Lysine 243 to Aspartic acid (K243D); and Lysine 386 to Arginine (K368R). (4B) a FASTA sequence of the rhAAT MJ6 variant (SEQ ID NO: 8). The specific mutations are indicated by a bold font and are underlined. (4C) Left, representative Ponceau S stain. Right, representative western blot using polyclonal anti-human-AAT antibodies. WT, WT-rhAAT. MJ6, MJ6-rhAAT. (4D) A representative Coomassie brilliant blue blot of: Lane 1, molecular weight standard; Lane 2, serum purified hAAT; Lane 3, purified recombinant hAAT (rhAAT) variant (MJ6 variant).

FIGS. 5A-5G include graphs showing anti-elastase inhibitory potency of hAAT mutants (mhAAT). The inhibitory potency over 0.39 μM of neutrophil elastase using any one of the following designated variants containing a single mutation: T22A (5A); T68A (5B); D74T (5C); D202H (5D); K243D (5E); and K368R (5F), and the MJ6 variant comprising all of the aforementioned mutations (5G). Wild type (WT) hAAT and serum-purified AAT (a commercially available Alpha1-Proteinase Inhibitor (Human)) were used as controls. Values represent mean±SEM.

FIGS. 6A-6C include vertical bar graphs showing the anti-inflammatory potency of mhAAT. Cell line of macrophagic lineage cells (RAW 264.7; 3×10⁵ cells per well) were incubated overnight with complete medium (RPMI 1640 containing 10% fetal bovine serum, 50 U/ml streptomycin/penicillin, 50 μg/ml L-glutamine) in the absence (−) or presence of 200 ng/ml of recombinant variants of rhAAT (T22A; T68A; D74T; D202H; K243D; K368R; MJ6; or WT) followed by PBS wash and re-incubation with complete medium containing LPS (5 ng/ml, 24 hr). (6A) Secreted LPS-induced IL-6 levels were measured by specific ELISA. (6B-6C) Flow cytometric analyses were performed to quantify the membranal activation markers, CD40^{Hi} (6B) and CD86^{Hi} (6C), gated of F4/80⁺ cells. Data represent 3 independent experimental repeats. Mean±SEM, *P<0.05, **P<0.01, ***P<0.001. CT—control, no LPS induction.

FIGS. 7A-7D include graphs showing the effects of the MJ6 variant on transcription of pro- and anti-inflammatory genes. Primary peritoneal macrophages (106 per well) were incubated overnight with complete medium containing 200 ng/ml wild type recombinant AAT (WT-rhAAT) or the MJ6 (MJ6-rhAAT) variant, followed by lipopolysaccharides (LPS) addition (10 ng/ml, 24 hr). Nucleic acids were extracted (0.5, 3, 6 hr) and transcription of IL-6 (7A); TNFα (7B); IL-10 (7C); and IL-1Ra (7D) were assessed by quantitative real-time PCR. Results presented as fold from control. CT—control, no LPS induction.

FIG. 8 includes a vertical bar graph showing a whole-blood anti-inflammatory assay. Fresh human blood was diluted 1:2 with complete medium. Samples were added with PBS, and: serum-purified AAT (0.5 mg/ml) or the MJ6 variant (200 ng/ml) and stimulated with LPS (10 ng/ml) two hours later. After 18 hours of incubation, all samples were centrifuged, supernatant were collected, and analyzed for human IL-6 using ELISA. Values represent mean±SEM, *p<0.05.

FIGS. 9A-9E include graphs showing in-vivo sterile inflammation model. C57BL/6 mice (n=3/group) were injected IP with serum purified AAT (sp-AAT) or the MJ6 variant (25 μg/mouse). Three hours post injection mice were injected with LPS (1 mg/kg). (9A) Peritoneal lavage was performed 24 hours post LPS injection and cells were stained for GR1 and F4/80 biomarkers. (9B-9E) Blood samples were obtained at 1.5, 3, and 24 hours post injection and analyzed for the levels of IL-6 (9B-9C) and TNFα (9D-9E) in the presence of either sp-AAT (9B and 9D) or the MJ6 variant (9C and 9E). CT—control, non-stimulated cells. Values represent mean±SEM.

### DETAILED DESCRIPTION

The present invention is directed to, in some embodiments, a mutant human alpha 1-antitrypsin (mhAAT) polypeptide, a pharmaceutical composition comprising same, and uses thereof.

In some embodiments, the present invention provides a polypeptide comprising the amino acid sequence: EDPQGDAAQKTDTSHHDQDHPTFNKITPNLAEFAFSLYRQLAHQSNSTNIFFSP VSIATAFAMLSLGTKADTHDEILEGLNFNLTEIPEAQIHEGFQELLHTLNQPDSQ LQLTTGNGLFLSEGLKLVDKFLEDVKKLYHSEAFTVNFGDTEEAKKQINDYVE KGTQGKIVDLVKELDRDTVFALVNYIFFKGKWERPFEVKDTEEEDFHVDQVTT VKVPMMKRLGMFNIQHCKKLSSWVLLMKYLGNATAIFFLPDEGKLQHLENEL THDIITKFLENEDRRSASLHLPKLSITGTYDLKSILGQLGITKVFSNGADLSGVTE EAPLKLSKAVHKAVLTIDEKGTEAAGAMFLEAIPMSIPPEVKFNKPFVFLMIEQN TKSPLFMGKVVNPTQK (SEQ ID NO: 9), wherein the polypeptide comprises at least one amino acid substitution at a position selected from: T22, T68, D74, D202, K243, and K368.

The terms “hAAT variant” and “mhAAT” are interchangeably used to refer to a nucleic acid and/or nucleotide sequences of hAAT comprising one or more substitutions. It should be appreciated that the wildtype sequence of hAAT (e.g., SEQ ID NO: 9), or analogs thereof comprising an amino acid substitution at a position C232, P357, or both, are not included under the scope of the present invention.

As used herein, the terms “peptide” and “polypeptide” are used interchangeably to refer to a polymer of amino acid residues. In some embodiments, a peptide or a polypeptide is a protein. In some embodiment, the peptide or polypeptide described herein comprise a modification rendering it more stable while in the body, more capable of penetrating into a cell or capable of eliciting a more potent effect than previously described. In some embodiment, the terms “peptide”, “polypeptide” and “protein” apply to naturally occurring amino acid polymers. In another embodiment, the terms “peptide”, “polypeptide” and “protein” apply to amino acid polymers in which one or more amino acid residue is an artificial chemical analogue of a corresponding naturally occurring amino acid.

In some embodiments, the polypeptide comprises at least 2, at least 3, at least 4, at least 5, or all 6 amino acid substitutions at a position selected from: T22, T68, D74, D202, K243, and K368, or any range therebetween. Each possibility represents a separate embodiment of the invention.

In some embodiments, the polypeptide of the invention comprises 6 amino acid substitutions at positions: T22, T68, D74, D202, K243, and K368.

In some embodiments, the polypeptide comprises 2 to 3, 2 to 4, 2 to 5, 2 to 6, 3 to 4, 3 to 5, 3 to 6, 4 to 5, 4 to 6, or 5 to 6 amino acid substitutions at a position selected from: T22, T68, D74, D202, K243, and K368. Each possibility represents a separate embodiment of the invention.

In some embodiments, the polypeptide is an isolated polypeptide.

As used herein, the term “isolated polypeptide” refers to a peptide that is essentially free from contaminating cellular components, such as carbohydrate, lipid, or other proteinaceous impurities associated with the peptide in nature. Typically, a preparation of isolated peptide contains the peptide in a highly-purified form, i.e., at least 80% pure, at least 90% pure, at least 95% pure, greater than 95% pure, or greater than 99% pure. Each possibility represents a separate embodiment of the invention.

In some embodiments, the polypeptide comprises the amino acid sequence: EDPQGDAAQKTDTSHHDQDHPX₁FNKITPNLAEFAFSLYRQLAHQSNSTNIFFSP VSIATAFAMLSLGX₂KADTHX₃EILEGLNFNLTEIPEAQIHEGFQELLHTLNQPDS QLQLTTGNGLFLSEGLKLVDKFLEDVKKLYHSEAFTVNFGDTEEAKKQINDYV EKGTQGKIVDLVKELDRDTVFALVNYIFFKGKWERPFEVKX₄TEEEDFHVDQVT TVKVPMMKRLGMFNIQHCKKLSSWVLLMX₅YLGNATAIFFLPDEGKLQHLENE LTHDIITKFLENEDRRSASLHLPKLSITGTYDLKSILGQLGITKVFSNGADLSGVT EEAPLKLSKAVHKAVLTIDEKGTEAAGAMFLEAIPMSIPPEVKFNX₆PFVFLMIE QNTKSPLFMGKVVNPTQK (SEQ ID NO: 1), wherein X₁ is T or A; X₂ is T or A; X₃ is D or T; X₄ is D or H; X₅ is K or D; and X₆ is K or R.

In some embodiments, the polypeptide comprises the amino acid sequence:

(SEQ ID NO: 8)
EDPQGDAAQKTDTSHHDQDHPAFNKITPNLAEFAFSLYRQLAHQSNSTNI
FFSPVSIATAFAMLSLGAKADTHTEILEGLNFNLTEIPEAQIHEGFQELL
HTLNQPDSQLQLTTGNGLFLSEGLKLVDKFLEDVKKLYHSEAFTVNFGDT
EEAKKQINDYVEKGTQGKIVDLVKELDRDTVFALVNYIFFKGKWERPFEV
KHTEEEDFHVDQVTTVKVPMMKRLGMFNIQHCKKLSSWVLLMDYLGNATA
IFFLPDEGKLQHLENELTHDIITKFLENEDRRSASLHLPKLSITGTYDLK
SILGQLGITKVFSNGADLSGVTEEAPLKLSKAVHKAVLTIDEKGTEAAGA
MFLEAIPMSIPPEVKFNRPFVFLMIEQNTKSPLFMGKVVNPTQK.

In some embodiments, the polypeptide comprises the amino acid sequence:

(SEQ ID NO: 2)
EDPQGDAAQKTDTSHHDQDHPAFNKITPNLAEFAFSLYRQLAHQSNSTNI
FFSPVSIATAFAMLSLGTKADTHDEILEGLNFNLTEIPEAQIHEGFQELL
HTLNQPDSQLQLTTGNGLFLSEGLKLVDKFLEDVKKLYHSEAFTVNFGDT
EEAKKQINDYVEKGTQGKIVDLVKELDRDTVFALVNYIFFKGKWERPFEV
KDTEEEDFHVDQVTTVKVPMMKRLGMFNIQHCKKLSSWVLLMKYLGNATA
IFFLPDEGKLQHLENELTHDIITKFLENEDRRSASLHLPKLSITGTYDLK
SILGQLGITKVFSNGADLSGVTEEAPLKLSKAVHKAVLTIDEKGTEAAGA
MFLEAIPMSIPPEVKFNKPFVFLMIEQNTKSPLFMGKVVNPTQK.

In some embodiments, the polypeptide comprises the amino acid sequence:

(SEQ ID NO: 3)
EDPQGDAAQKTDTSHHDQDHPTFNKITPNLAEFAFSLYRQLAHQSNSTNI
FFSPVSIATAFAMLSLGAKADTHDEILEGLNFNLTEIPEAQIHEGFQELL
HTLNQPDSQLQLTTGNGLFLSEGLKLVDKFLEDVKKLYHSEAFTVNFGDT
EEAKKQINDYVEKGTQGKIVDLVKELDRDTVFALVNYIFFKGKWERPFEV
KDTEEEDFHVDQVTTVKVPMMKRLGMFNIQHCKKLSSWVLLMKYLGNATA
IFFLPDEGKLQHLENELTHDIITKFLENEDRRSASLHLPKLSITGTYDLK
SILGQLGITKVFSNGADLSGVTEEAPLKLSKAVHKAVLTIDEKGTEAAGA
MFLEAIPMSIPPEVKFNKPFVFLMIEQNTKSPLFMGKVVNPTQK.

In some embodiments, the polypeptide comprises the amino acid sequence:

(SEQ ID NO: 4)
EDPQGDAAQKTDTSHHDQDHPTFNKITPNLAEFAFSLYRQLAHQSNSTNI
FFSPVSIATAFAMLSLGTKADTHTEILEGLNFNLTEIPEAQIHEGFQELL
HTLNQPDSQLQLTTGNGLFLSEGLKLVDKFLEDVKKLYHSEAFTVNFGDT
EEAKKQINDYVEKGTQGKIVDLVKELDRDTVFALVNYIFFKGKWERPFEV
KDTEEEDFHVDQVTTVKVPMMKRLGMFNIQHCKKLSSWVLLMKYLGNATA
IFFLPDEGKLQHLENELTHDIITKFLENEDRRSASLHLPKLSITGTYDLK
SILGQLGITKVFSNGADLSGVTEEAPLKLSKAVHKAVLTIDEKGTEAAGA
MFLEAIPMSIPPEVKFNKPFVFLMIEQNTKSPLFMGKVVNPTQK.

In some embodiments, the polypeptide comprises the amino acid sequence:

(SEQ ID NO: 5)
EDPQGDAAQKTDTSHHDQDHPTFNKITPNLAEFAFSLYRQLAHQSNSTNI
FFSPVSIATAFAMLSLGTKADTHDEILEGLNFNLTEIPEAQIHEGFQELL
HTLNQPDSQLQLTTGNGLFLSEGLKLVDKFLEDVKKLYHSEAFTVNFGDT
EEAKKQINDYVEKGTQGKIVDLVKELDRDTVFALVNYIFFKGKWERPFEV
KHTEEEDFHVDQVTTVKVPMMKRLGMFNIQHCKKLSSWVLLMKYLGNATA
IFFLPDEGKLQHLENELTHDIITKFLENEDRRSASLHLPKLSITGTYDLK
SILGQLGITKVFSNGADLSGVTEEAPLKLSKAVHKAVLTIDEKGTEAAGA
MFLEAIPMSIPPEVKFNKPFVFLMIEQNTKSPLFMGKVVNPTQK.

In some embodiments, the polypeptide comprises the amino acid sequence:

(SEQ ID NO: 6)
EDPQGDAAQKTDTSHHDQDHPTFNKITPNLAEFAFSLYRQLAHQSNSTNI
FFSPVSIATAFAMLSLGTKADTHDEILEGLNFNLTEIPEAQIHEGFQELL
HTLNQPDSQLQLTTGNGLFLSEGLKLVDKFLEDVKKLYHSEAFTVNFGDT
EEAKKQINDYVEKGTQGKIVDLVKELDRDTVFALVNYIFFKGKWERPFEV
KDTEEEDFHVDQVTTVKVPMMKRLGMFNIQHCKKLSSWVLLMDYLGNATA
IFFLPDEGKLQHLENELTHDIITKFLENEDRRSASLHLPKLSITGTYDLK
SILGQLGITKVFSNGADLSGVTEEAPLKLSKAVHKAVLTIDEKGTEAAGA
MFLEAIPMSIPPEVKFNKPFVFLMIEQNTKSPLFMGKVVNPTQK.

In some embodiments, the polypeptide comprises the amino acid sequence:

(SEQ ID NO: 7)
EDPQGDAAQKTDTSHHDQDHPTFNKITPNLAEFAFSLYRQLAHQSNSTNI
FFSPVSIATAFAMLSLGTKADTHDEILEGLNFNLTEIPEAQIHEGFQELL
HTLNQPDSQLQLTTGNGLFLSEGLKLVDKFLEDVKKLYHSEAFTVNFGDT
EEAKKQINDYVEKGTQGKIVDLVKELDRDTVFALVNYIFFKGKWERPFEV
KDTEEEDFHVDQVTTVKVPMMKRLGMFNIQHCKKLSSWVLLMKYLGNATA
IFFLPDEGKLQHLENELTHDIITKFLENEDRRSASLHLPKLSITGTYDLK
SILGQLGITKVFSNGADLSGVTEEAPLKLSKAVHKAVLTIDEKGTEAAGA
MFLEAIPMSIPPEVKFNRPFVFLMIEQNTKSPLFMGKVVNPTQK.

In some embodiments, the present invention is further directed to an analog and/or a chemically modified form (“derivative”) of the polypeptide of the invention, as long as they are capable of excreting the anti-inflammatory activity attributed to the polypeptide of the invention, as disclosed hereinbelow.

The term “analog” includes any peptide having an amino acid sequence substantially identical to one of the sequences specifically shown herein in which one or more residues have been conservatively substituted with a functionally similar residue and which displays the abilities as described herein. Examples of conservative substitutions include the substitution of one non-polar (hydrophobic) residue such as isoleucine, valine, leucine or methionine for another, the substitution of one polar (hydrophilic) residue for another such as between arginine and lysine, between glutamine and asparagine, between glycine and serine, the substitution of one basic residue such as lysine, arginine or histidine for another, or the substitution of one acidic residue, such as aspartic acid or glutamic acid for another. Each possibility represents a separate embodiment of the present invention.

In some embodiments, the analog is a functional analog.

As used herein, “a functional analog” refers to a polypeptide analogous to the polypeptide of the invention and characterized by having essentially the same activity as the polypeptide of the invention, as described herein.

In some embodiments, essentially the same is at least 90%, 93%, 95%, 96%, 97%, 98%, 99%, or 100% compared to the polypeptide of the invention, or any value and range therebetween. Each possibility represents a separate embodiment of the invention. In some embodiments, essentially the same is 90% to 95%, 92% to 98%, 90% to 99%, 90% to 100%, 94% to 99%, or 95% to 100%, compared to the polypeptide of the invention. Each possibility represents a separate embodiment of the invention.

The phrase “conservative substitution” also includes the use of a chemically derivatized residue in place of a non-derivatized residue provided that such peptide displays the requisite function of modulating the immune system's innate response as specified herein.

The term “derivative” or “chemical derivative” includes any chemical derivative of the polypeptide having one or more residues chemically derivatized by reaction of side chains or functional groups. Such derivatized molecules include, for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Also included as chemical derivatives are those polypeptides, which contain one or more naturally occurring amino acid derivatives of the twenty standard amino acid residues. For example: 4-hydroxyproline may be substituted for proline; 5-hydroxylysine may be substituted for lysine; 3-methylhistidine may be substituted for histidine; homoserine may be substituted or senne; and ornithine may be substituted for lysine.

The term “derived from” or “corresponding to” refers to construction of an amino acid sequence based on the knowledge of a sequence using any one of the suitable means known to one skilled in the art, e.g., chemical synthesis in accordance with standard protocols in the art.

In addition, a polypeptide derivative can differ from the natural sequence of the polypeptide of the invention by chemical modifications including, but are not limited to, terminal-NH₂ acylation, acetylation, or thioglycolic acid amidation, and by terminal-carboxlyamidation, e.g., with ammonia, methylamine, and the like. Peptides can be either linear, cyclic or branched and the like, which conformations can be achieved using methods well known in the art.

The polypeptide derivatives according to the principles of the present invention can also include side chain bond modifications, including but not limited to —CH2-NH—, —CH2-S—, —CH2-S═O, OC—NH—, —CH2-O—, —CH2-CH2-, S═C—NH—, and —CH═CH—, and backbone modifications such as modified peptide bonds. Peptide bonds (—CO—NH—) within the peptide can be substituted, for example, by N-methylated bonds (—N(CH3)-CO—); ester bonds (—C(R)H—C—O—O—C(R)H—N); ketomethylene bonds (—CO—CH2-); a-aza bonds (—NH—N(R)—CO—), wherein R is any alkyl group, e.g., methyl; carba bonds (—CH2-NH—); hydroxyethylene bonds (—CH(OH)—CH2-); thioamide bonds (—CS—NH); olefinic double bonds (—CH═CH—); and peptide derivatives (—N(R)—CH2-CO—), wherein R is the “normal” sidechain, naturally presented on the carbon atom. These modifications can occur at one or more of the bonds along the peptide chain and even at several (e.g., 2-3) at the same time.

In some embodiments, the polypeptide derivative contains non-natural amino acids. Examples of non-natural amino acids include, but are not limited to, sarcosine (Sar), norleucine, ornithine, citrulline, diaminobutyric acid, homoserine, isopropyl Lys, 3-(2′-naphtyl)-Ala, nicotinyl Lys, amino isobutyric acid, and 3-(3′-pyridyl-Ala).

In some embodiments, the polypeptide derivative contains other derivatized amino acid residues. Examples of derivatized amino acid residues include, but are not limited to, methylated amino acids, N-benzylated amino acids, O-benzylated amino acids, N-acetylated amino acids, O-acetylated amino acids, carbobenzoxy-substituted amino acids and the like. Specific examples include, but are not limited to, methyl-Ala (Me Ala), MeTyr, MeArg, MeGlu, MeVal, MeHis, N-acetyl-Lys, O-acetyl-Lys, carbobenzoxy-Lys, Tyr-O-Benzyl, Glu-O-Benzyl, Benzyl-His, Arg-Tosyl, t-butylglycine, t-butylalanine, phenylglycine, cyclohexylalanine, and the like.

In some embodiments, the invention is further directed to a polypeptide analog, which contains one or more D-isomer forms of the amino acids. Production of retro-inverso D-amino acid peptides where at least one amino acid, and perhaps all amino acids are D-amino acids is well known in the art. When all of the amino acids in the peptide are D-amino acids, and the N- and C-terminals of the molecule are reversed, the result is a molecule having the same structural groups being at the same positions as in the L-amino acid form of the molecule. However, the molecule is more stable to proteolytic degradation and is therefore useful in many of the applications recited herein. Diastereomeric peptides may be highly advantageous over all L- or all D-amino acid peptides having the same amino acid sequence because of their higher water solubility, lower immunogenicity, and lower susceptibility to proteolytic degradation. The term “diastereomeric peptide” as used herein refers to a peptide comprising both L-amino acid residues and D-amino acid residues. The number and position of D-amino acid residues in a diastereomeric peptide of the preset invention may be variable so long as the peptide is capable of displaying the requisite function, e.g., anti-inflammatory activity, as specified herein.

The term “analog” and “derivative” are used herein interchangeably.

The polypeptide of the invention may be synthesized or prepared by techniques well known in the art. The polypeptide can be synthesized by a solid phase peptide synthesis method of Merrifield (see J. Am. Chem. Soc, 85:2149, 1964). Alternatively, the polypeptide of the present invention can be synthesized using standard solution methods well known in the art (see, for example, Bodanszky, M., Principles of Peptide Synthesis, Springer-Verlag, 1984) or by any other method known in the art for peptide synthesis.

In general, the aforementioned methods comprise sequential addition of one or more amino acids or suitably protected amino acids to a growing peptide chain bound to a suitable resin.

Normally, either the amino or carboxyl group of the first amino acid is protected by a suitable protecting group. The protected or derivatized amino acid can then be either attached to an inert solid support (resin) or utilized in solution by adding the next amino acid in the sequence having the complimentary (amino or carboxyl) group suitably protected, under conditions conductive for forming the amide linkage. The protecting group is then removed from this newly added amino acid residue and the next amino acid (suitably protected) is added, and so forth. After all the desired amino acids have been linked in the proper sequence, any remaining protecting groups are removed sequentially or concurrently, and the peptide chain, if synthesized by the solid phase method, is cleaved from the solid support to afford the final peptide.

In the solid phase peptide synthesis method, the alpha-amino group of the amino acid is protected by an acid or base sensitive group. Such protecting groups should have the properties of being stable to the conditions of peptide linkage formation, while being readily removable without destruction of the growing peptide chain. Suitable protecting groups are t-butyloxycarbonyl (BOC), benzyloxycarbonyl (Cbz), biphenylisopropyloxycarbonyl, t-amyloxycarbonyl, isobornyloxycarbonyl, (alpha, alpha)-dimethyl-3,5dimethoxybenzyloxycarbonyl, o-nitrophenylsulfenyl, 2-cyano-t-butyloxycarbonyl, 9-fluorenylmethyloxycarbonyl (FMOC) and the like.

In the solid phase peptide synthesis method, the C-terminal amino acid is attached to a suitable solid support. Suitable solid supports useful for the above synthesis are those materials, which are inert to the reagents and reaction conditions of the stepwise condensation-deprotection reactions, as well as being insoluble in the solvent media used. Suitable solid supports are chloromethylpolystyrene-divinylbenzene polymer, hydroxymethyl-polystyrene-divinylbenzene polymer, and the like. The coupling reaction is accomplished in a solvent such as ethanol, acetonitrile, N,N-dimethylformamide (DMF), and the like. The coupling of successive protected amino acids can be carried out in an automatic polypeptide synthesizer as is well known in the art.

The polypeptide of the invention may alternatively be synthesized such that one or more of the bonds, which link the amino acid residues of the polypeptide are non-peptide bonds. These alternative non-peptide bonds include, but are not limited to, imino, ester, hydrazide, semicarbazide, and azo bonds, which can be formed by reactions well known to skilled in the art.

In some embodiments, recombinant protein techniques are used to generate the polypeptide of the invention. In some embodiments, recombinant protein techniques are used for generation of relatively long peptides (e.g., longer than 18-25 amino acids). In some embodiments, recombinant protein techniques are used for the generation of large amounts of the polypeptide of the invention. In some embodiments, recombinant techniques are described by Bitter et al., (1987) Methods in Enzymol. 153:516-544, Studier et al. (1990) Methods in Enzymol. 185:60-89, Brisson et al. (1984) Nature 310:511-514, Takamatsu et al. (1987) EMBO J. 6:307-311, Coruzzi et al. (1984) EMBO J. 3:1671-1680 and Brogli et al, (1984) Science 224:838-843, Gurley et al. (1986) Mol. Cell. Biol. 6:559-565 and Weissbach & Weissbach, 1988, Methods for Plant Molecular Biology, Academic Press, NY, Section VIII, pp 421-463.

Any one of the polypeptides of the present invention, an analog thereof, and a derivative thereof, produced by recombinant techniques can be purified so that the polypeptide will be substantially pure when administered to a subject. The term “substantially pure” refers to a compound, e.g., a polypeptide, which has been separated from components, which naturally accompany it.

Typically, a polypeptide is substantially pure when at least 50%, at least 75%, at least 90%, and at least 99% of the total material (by volume, by wet or dry weight, or by mole percent or mole fraction) in a sample is the polypeptide of interest, or any value and range therebetween. Each possibility represents a separate embodiment of the invention. Purity can be measured by any appropriate method, e.g., in the case of peptides by HPLC analysis.

In some embodiments, there is provided a polynucleotide sequence encoding the polypeptide of the present invention, an analog or a derivative thereof. In some embodiments, the polynucleotide encodes an amino acid sequence comprising any one of SEQ ID Nos.: 1-8. It is within the capabilities of a skilled artisan to generate the mhAAT variants of the invention by introducing the herein disclosed substitutions into a polynucleotide template comprising the wild type hATT, as set forth is the sequence:

(SEQ ID NO: 23)
ACATGTAATCGACAATGCCGTCTTCTGTCTCGTGGGGCATCCTCCTGGCA
GGCCTGTGCTGCCTGGTCCCTGTCTCCCTGGCTGAGGATCCCCAGGGAGA
TGCTGCCCAGAAGACAGATACATCCCACCATGATCAGGATCACCCAACCT
TCAACAAGATCACCCCCAACCTGGCTGAGTTCGCCTTCAGCCTATACCGC
CAGCTGGCACACCAGTCCAACAGCACCAATATCTTCTTCTCCCCAGTGAG
CATCGCTACAGCCTTTGCAATGCTCTCCCTGGGGACCAAGGCTGACACTC
ACGATGAAATCCTGGAGGGCCTGAATTTCAACCTCACGGAGATTCCGGAG
GCTCAGATCCATGAAGGCTTCCAGGAACTCCTCCGTACCCTAAACCAGCC
AGACAGCCAGCTCCAGCTGACCACCGGCAATGGCCTGTTCCTCAGCGAGG
GCCTGAAGCTAGTGGATAAGTTTTTGGAGGATGTTAAAAAGTTGTACCAC
TCAGAAGCCTTCACTGTCAACTTCGGGGATCACGAAGAGGCCAAGAAACA
GATCAACGATTACGTGGAGAAGGGTACTCAAGGGAAAATTGTGGATTTGG
TCAAGGAGCTTGACAGAGACACAGTTTTTGCTCTGGTGAATTACATCTTC
TTTAAAGGCAAATGGGAGAGACCTTTTGAAGTCAAGGACACCGAGGACGA
GGACTTCCACGTGGACCAGGTGACCACCGTGAAGGTCCCTATGATGAAGC
GTTTAGGCATGTTTAACATCCAGCACTGTAAGAAGCTGTCCAGCTGGGTA
CTGCTAATGAAATACCTGGGCAATGCCACCGCCATCTTCTTCCTACCTGA
TGAGGGGAAACTACAGCACCTGGAAAATGAACTCACCCACGATATCATCA
CCAAGTTCCTGGAAAATGAAGACAGAAGGTCTGCCAGCTTACATTTACCC
AAACTGTCCATTACTGGAACCTATGATCTGAAGAGCGTCCTGGGTCAACT
GGGCATCACTAAGGTCTTCAGCAATGGGGCTGACCTCTCCGGGGTCACAG
AGGAGGCACCCCTGAAGCTCTCCAAGGCCGTGCATAAGGCTGTGCTGACC
ATCGACGAGAAGGGGACTGAAGCTGCTGGGGCCATGTTTTTAGAGGCCAT
ACCAATGTCTATCCCCCCAGAGGTCAAGTTCAACAAACCCTTTGTCTTCT
TAATGATTGAACAAAATACCAAGTCTCCCCTCTTCATGGGAAAAGTGGTG
AATCCCACCCAAAAATAACTGCCTCTCGCTCCTCAACCCCTCCCCTCCAT
CCCTGGCCCCCTCCCTGGATGACATTAAAGAAGGGTTGAGCTGGA.

As used herein, the term “polynucleotide” refers to a polymer of deoxyribonucleic acid (DNA), ribonucleic acid (RNA) or a combination thereof, which can be derived from any source, can be single-stranded or double-stranded, and can optionally contain synthetic, non-natural, or altered nucleotides, which are capable of being incorporated into DNA or RNA polymers.

In some embodiments, the polynucleotide is an isolated polynucleotide.

As used herein, the term “isolated polynucleotide” refers to a polynucleotide segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to a polynucleotide, which has been substantially purified from other components, which naturally accompany the polynucleotide in a cell, e.g., RNA or DNA or proteins. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA, which is part of a hybrid gene encoding additional polypeptide sequence, and RNA such as mRNA.

As used herein, the term “encoding” refers to the inherent property of specific sequences of nucleotides in an isolated polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a peptide or protein if transcription and translation of mRNA corresponding to that gene produces the peptide or protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the peptide or protein or other product of that gene or cDNA.

One who is skilled in the art will appreciate that more than one polynucleotide may encode any given polypeptide or protein in view of the degeneracy of the genetic code and the allowance of exceptions to classical base pairing in the third position of the codon, as given by the so-called “Wobble rules”. It is intended that the present invention encompass polynucleotides that encode the polypeptide of the invention as well as any analog thereof, or derivative thereof.

In some embodiments, the polynucleotide is expressed and the polypeptide of the invention, the analog thereof, or the derivative thereof, is secreted, e.g., from a host cell to a culture media. In some embodiments, the secreted polypeptide is isolated from the medium in which the host cell containing the polynucleotide is cultured, or the polynucleotide can be expressed as an intracellular polypeptide by deleting the signal peptide or other related peptides, in which case the polypeptide of the invention, the analog thereof, or the derivative thereof, is isolated from the host cell. In some embodiments, the polypeptide of the invention the analog thereof, or the derivative thereof, is purified by standard protein purification methods known in the art.

In some embodiments, there is provided an artificial vector comprising the polynucleotide of the invention.

In some embodiments, the artificial vector comprises or is an expression vector.

The expression vector according to the principles of the present invention further comprises a promoter. In the context of the present invention, the promoter must be able to drive the expression of the polypeptide within the cell. Many viral promoters are appropriate for use in such an expression vector (e.g., retroviral ITRs, LTRs, immediate early viral promoters (IEp) (such as herpes virus IEp (e.g., ICP4-IEp and ICPO-IEp) and cytomegalovirus (CMV) IEp), and other viral promoters (e.g., late viral promoters, latency-active promoters (LAPs), Rous Sarcoma Virus (RSV) promoters, and Murine Leukemia Virus (MLV) promoters). Other suitable promoters are eukaryotic promoters, which contain enhancer sequences (e.g., the rabbit β-globin regulatory elements), constitutively active promoters (e.g., the β-actin promoter, etc.), signal and/or tissue specific promoters (e.g., inducible and/or repressible promoters, such as a promoter responsive to TNF or RU486, the metallothionine promoter, etc.), and tumor-specific promoters.

Within the expression vector, the polynucleotide encoding the polypeptide of the invention, an analog thereof, or a derivative thereof, and the promoter are operably linked such that the promoter is able to drive the expression of the polynucleotide. As long as this operable linkage is maintained, the expression vector can include more than one gene, such as multiple genes separated by internal ribosome entry sites (IRES). Furthermore, the expression vector can optionally include other elements, such as splice sites, polyadenylation sequences, transcriptional regulatory elements (e.g., enhancers, silencers, etc.), or other sequences.

The expression vector is introduced into the cell in a manner such that it is capable of expressing the polynucleotide encoding the polypeptide of the invention, an analog thereof, or derivative thereof, contained therein. Any suitable vector can be so employed, many of which are known in the art. Non-limiting examples of such vectors include naked DNA vectors (such as oligonucleotides or plasmids), viral vectors such as adeno-associated viral vectors (Berns et al, 1995, Ann. N.Y. Acad. Sci. 772:95-104, the contents of which are hereby incorporated by reference in their entirety), adenoviral vectors, herpes virus vectors (Fink et al, 1996, Ann. Rev. Neurosci. 19:265-287), packaged amplicons (Federoff et al, 1992, Proc. Natl. Acad. Sci. USA 89: 1636-1640, the contents of which are hereby incorporated by reference in their entirety), papilloma virus vectors, picomavirus vectors, polyoma virus vectors, retroviral vectors, SV40 viral vectors, vaccinia virus vectors, and other vectors. Additionally, the vector can also include other genetic elements, such as, for example, genes encoding a selectable marker (e.g., β-gal or a marker conferring resistance to a toxin), a pharmacologically active protein, a transcription factor, or other biologically active substance. Thus, in the case of prokaryotic cells, vector introduction can be accomplished, for example, by electroporation, transformation, transduction, conjugation, or mobilization. For eukaryotic cells, vectors can be introduced through the use of, for example, electroporation, transfection, infection, DNA coated microprojectiles, or protoplast fusion. Examples of eukaryotic cells into which the expression vector can be introduced include, but are not limited to, ovum, stem cells, blastocytes, and the like.

Methods for manipulating a vector comprising a polynucleotide are well known in the art (e.g., Sambrook et al., 1989, Molecular Cloning: A Laboratory Manual, 2nd edition, Cold Spring Harbor Press, the contents of which are hereby incorporated by reference in their entirety) and include direct cloning, site specific recombination using recombinases, homologous recombination, and other suitable methods of constructing a recombinant vector. In this manner, an expression vector can be constructed such that it can be replicated in any desired cell, expressed in any desired cell, and can even become integrated into the genome of any desired cell.

In some embodiments, mammalian expression vectors include, but are not limited to, pcDNA3, pcDNA3.1 (±), pGL3, pZeoSV2(±), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

In some embodiments, expression vectors containing regulatory elements from eukaryotic viruses such as retroviruses are used by the present invention. SV40 vectors include pSVT7 and pMT2. In some embodiments, vectors derived from bovine papilloma virus include pBV-1MTHA, and vectors derived from Epstein Bar virus include pHEBO, and p2O5. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

In some embodiments, recombinant viral vectors, which offer advantages such as lateral infection and targeting specificity, are used for in vivo expression. In one embodiment, lateral infection is inherent in the life cycle of, for example, retrovirus and is the process by which a single infected cell produces many progeny virions that bud off and infect neighboring cells. In one embodiment, the result is that a large area becomes rapidly infected, most of which was not initially infected by the original viral particles. In one embodiment, viral vectors are produced that are unable to spread laterally. In one embodiment, this characteristic can be useful if the desired purpose is to introduce a specified gene into only a localized number of targeted cells.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

In one embodiment, plant expression vectors are used. In one embodiment, the expression of a polypeptide coding sequence is driven by a number of promoters. In some embodiments, viral promoters such as the 35S RNA and 19S RNA promoters of CaMV [Brisson et al., Nature 310:511-514 (1984)], or the coat protein promoter to TMV [Takamatsu et al., EMBO J. 6:307-311 (1987)] are used. In another embodiment, plant promoters are used such as, for example, the small subunit of RUBISCO [Coruzzi et al., EMBO J. 3:1671-1680 (1984); and Brogli et al., Science 224:838-843 (1984)] or heat shock promoters, e.g., soybean hspl7.5-E or hsp17.3-B [Gurley et al., Mol. Cell. Biol. 6:559-565 (1986)]. In one embodiment, constructs are introduced into plant cells using Ti plasmid, Ri plasmid, plant viral vectors, direct DNA transformation, microinjection, electroporation and other techniques well known to the skilled artisan. See, for example, Weissbach & Weissbach [Methods for Plant Molecular Biology, Academic Press, NY, Section VIII, pp 421-463 (1988)]. Other expression systems such as insects and mammalian host cell systems, which are well known in the art, can also be used by the present invention.

It will be appreciated that other than containing the necessary elements for the transcription and translation of the inserted coding sequence (encoding the polypeptide), the expression construct of the present invention can also include sequences engineered to optimize stability, production, purification, yield or activity of the expressed polypeptide.

In some embodiments, a cell comprising any one of: the polypeptide of the invention, an analog thereof, a derivative thereof, and a polynucleotide encoding thereof, is provided. In some embodiments, the cell comprises the artificial vector disclosed herein.

In some embodiments, a cell into which the polynucleotide has been transferred under the control of an inducible promoter if necessary, can be used as a transient transformant. Such a cell may then be transferred into a subject for therapeutic benefit therein.

Within the cell, the polynucleotide encoding the polypeptide of the invention, an analog thereof, or a derivative thereof, is expressed, and optionally is secreted therefrom. Successful expression of the polynucleotide can be assessed using standard molecular biology techniques (e.g., Northern hybridization, Western blotting, immunoprecipitation, enzyme immunoassay, etc.).

In some embodiments, there is provided a composition comprising: (a) the polypeptide of the invention, an analog thereof, or a derivative thereof; (b) a polynucleotide encoding the polypeptide of the invention; (c) an artificial vector comprising the polynucleotide; (d) a cell comprising (a), (b), (c), or any combination thereof, (e) any combination of (a), (b), (c), and (d); and an acceptable carrier.

According to some embodiments, there is provided a pharmaceutical composition comprising any of the polypeptides of the invention and a pharmaceutically acceptable carrier, excipient, or adjuvant.

In some embodiments, the pharmaceutical composition is for use in the treatment or prevention of a condition selected from: AAT deficiency, an AAT related disease, and inflammation, in a subject in need thereof.

In some embodiments, the pharmaceutical composition is for use in treating a subject in need of alpha 1-antitrypsin therapy.

As used herein, the term “carrier,” or “excipient” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8^{th} Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21St Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptide or polypeptide in serum. Liposomes include emulsions, foams, micelles, insoluble monolayers, liquid crystals, phospholipid dispersions, lamellar layers and the like. Liposomes for use with the presently described peptide are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

In some embodiments, the carrier may comprise, in total, from about 0.1% to about 99.99999% by weight of the pharmaceutical compositions presented herein.

In some embodiments, the pharmaceutical composition may take any physical form necessary for proper administration. The composition may be administered orally in the form of a pill, capsule or liquid. The composition may be in the form of a gel, spray, cream or ointment.

According to some embodiments, there is provided a method for treating, ameliorating, or preventing a condition selected from: AAT deficiency, an AAT related disease, and inflammation, in a subject in need thereof, comprising administering to the subject a therapeutically effective amount of any one of: (i) the polypeptide of the invention; and (ii) a pharmaceutical composition comprising thereof, thereby treating the subject afflicted with the condition selected from: AAT deficiency, an AAT related disease, and inflammation.

According to some embodiments, there is provided a method for modulating the activity of an immune cell, comprising contacting the immune cell with an effective amount of the polypeptide of the invention, thereby modulating the activity of the immune cell.

According to some embodiments, there is provided a method of treating a subject in need of alpha 1- anti trypsin therapy, comprising administering to the subject a therapeutically effective amount of any of: (i) the polypeptide of the invention; and (ii) a pharmaceutical composition comprising thereof, thereby treating a subject in need of alpha 1-antitrypsin therapy.

In some embodiments, the activity of an immune cells is selected from: anti-inflammatory activity, protease inhibiting activity, pro-inflammatory activity, or any combination thereof.

As used herein, the terms “inflammatory” or “inflammation” refer to a complex protective reaction or a part thereof, which may involve immune cells, blood vessels, and molecular mediators, exerted by an organism or a tissue thereof in response to a stimulus, including, but not limited to, a pathogen or a damaged cell. The main purpose of the inflammatory process is to remove or clear the causative agent and initiate tissue repair.

As used herein, the term “AAT deficiency” refers to a genetic disorder, e.g., a mutation in the SERPINA1 gene. The mutation results in insufficient levels of alpha-1 antitrypsin which may in turn lead to a liver disease or a lung disease. The terms “AAT deficiency”, “A1AD”, and “AATD” are interchangeable. Diagnosing AAT deficiency is well within the capabilities of one of ordinary skill in the art. Non-limiting examples for methods of diagnosing AATD include, but are not limited to, immunoassays for determining serum alpha-1 antitrypsin levels (e.g., ELISA), and DNA sequencing/genotyping for determining the presence of a mutation in the SERPINA1 gene.

As used herein, the term “AAT related disease” encompasses any condition or disease in which AAT is involved in the pathogenesis, pathophysiology, or both. In some embodiments, AAT involvement in an AAT related disease is AAT hypoactivity. In some embodiments, AAT involvement in an AAT related disease is AAT hyperactivity.

Non-limiting examples of AAT related diseases or conditions include but are not limited to genetic alpha 1-antitrypsin deficiency, emphysema, chronic obstructive pulmonary disease (COPD), bronchiectasis, parenchymatic and fibrotic lung diseases or disorders, cystic fibrosis, interstitial pulmonary fibrosis, lung sarcoidosis, liver cirrhosis, liver failure, tuberculosis and lung diseases and disorders secondary to HIV.

As used herein, the phrases “protease inhibiting” or “protease inhibitor” encompasses any agent which reduces, inhibits, eliminates, hampers, decreases, or any equivalent thereof, of the activity of a protease or a proteolytic enzyme. As used herein the term “protease” encompasses any enzyme or agent which is capable of hydrolyzing a peptide bond. As used herein, the terms “protease”, “peptidase” and “proteinase” are interchangeable.

In some embodiments, the protease is elastase. In some embodiments, elastase is a neutrophil elastase.

In some embodiments, the term “modulating” is altering. In another embodiment, the term “modulating” is activating. In another embodiment, the term “modulating” is inhibiting. In another embodiment, the term “modulating” is increasing. In another embodiment, the term “modulating” is inducing. In another embodiment, the term “modulating” is elevating. In another embodiment, the term “modulating” is reducing. In another embodiment, the term “modulating” is differentially activating. In another embodiment, the term “modulating” is decreasing. In another embodiment, the term “modulating” is differentially inhibiting. In another embodiment, modulating an immune response includes the activation and/or induction of certain immune cells or sub-sets, while at the same time inhibiting other immune cells or particular sub-sets immune cells.

In some embodiments, the polypeptide of the invention, or a composition comprising thereof has an anti-inflammatory activity.

In some embodiments, the method of the invention comprises activating, initiating, promoting, propagating, inducing, or any equivalent thereof, an anti-inflammatory activity of an immune cell.

In some embodiments, the method of the invention comprises activating, initiating, promoting, propagating, inducing, or any equivalent thereof, an anti-inflammatory response in the subject.

In some embodiments, the method of the invention comprises preventing an inflammatory response in the subject.

In some embodiments, the polypeptide of the invention, or a composition comprising thereof inhibits or reduces an inflammatory activity.

In some embodiments, the method comprises inhibiting or reducing a pro-inflammatory activity of an immune cell.

In some embodiments, the method of the invention comprises inhibiting or reducing, an inflammatory response in the subject.

In some embodiments, the polypeptide of the invention, or a composition comprising thereof inhibits or reduces a protease activity. In some embodiments, the polypeptide of the invention, or a composition comprising thereof inhibits or reduces cellular proteolysis levels or rates. Method for quantifying proteolytic activity and/or inhibition thereof are known in the art, and may include, but are not limited to protease inhibition assay, such as exemplified herein below.

In some embodiments, contacting is contacting in vivo or in vitro. In some embodiments, contacting is contacting in vivo and in vitro.

As used herein the term “in vitro” refers to any process that occurs outside a living organism. As used herein the term “in-vivo” refers to any process that occurs inside a living organism.

In some embodiments, any one of the polypeptide of the invention and a pharmaceutical composition comprising thereof reduce the abundance of a CD40^{Hi} cell, a CD86^{Hi} cell, or both, in the subject.

Methods for quantifying cell surface markers are common and would be apparent to one of ordinary skill in the art. A non-limiting example for a method of quantifying cell surface marker includes but is not limited to fluorescence activated cell sorting (FACS) using specific antibodies, as exemplified herein below.

In some embodiments, any one of the polypeptide of the invention and a pharmaceutical composition comprising thereof reduce the expression level, the secretion level, or both, of a factor selected from interleukin (IL)-6, and tumor necrosis factor alpha, in the subject.

In some embodiments, any one of the polypeptide of the invention and a pharmaceutical composition comprising thereof increase the expression level, the secretion level, or both, of a factor selected from IL-10, and IL-1Ra, in the subject.

In some embodiments, increased is by at least 5%, by at least 15%, by at least 30%, by at least 50%, by at least 75%, by at least 100%, by at least 150%, by at least 250%, by at least 400%, by at least 550%, by at least 750%, or by at least 1,000% more, compared to control, or any value and range therebetween. Each possibility represents a separate embodiment of the invention. In some embodiments, increased is by 1-50%, 5-250%, 75-450%, 100-650%, 175-875%, 10-915%, or 100-1,000% more compared to control. Each possibility represents a separate embodiment of the invention.

As used herein, the terms “reduce” or “reducing” encompass eliminating, omitting, inhibiting, depriving, or any equivalent thereof.

In some embodiments reducing is by at least 5%, at least 10%, at least 15%, at least 25%, at least 30%, at least 40%, at least 50%, at least 65%, at least 75%, at least 80%, at least 85%, at least 90%, at least 95%, or at least 99% compared to control, or any value and range therebetween. Each possibility represents a separate embodiment of the invention. In some embodiments reducing is by 1-20%, 5-35%, 9-55%, 2-65%, 1-75%, 20-90%, 65-99%, 20-100% compared to control. Each possibility represents a separate embodiment of the invention.

In some embodiments, a control is a healthy subject. In some embodiments, a control is non-treated subject. In some embodiments, a control is a wild type hAAT. In some embodiments, a control is mhAAT variant devoid of one or more of the herein disclosed amino acid substitutions (as specified for SEQ ID NO: 1).

Methods for determining gene expression and/or protein secretion are common and would be apparent to one of ordinary skill in the art of molecular biology and biochemistry. Non-limiting examples of methods for determining gene expression levels, include, but are not limited to, next generation sequencing, PCR, and quantitative PCR (qPCR) as exemplified herein below. Methods for determining secretion levels of a polypeptide or any proteinaceous factor include, but are not limited to immunoassays, such as western-blot, dot-blot, and ELISA as exemplified herein below.

In some embodiments, an immune cell is a neutrophil or a macrophage.

In some embodiments, the immune cell is a cell of a subject.

In some embodiments, the subject is in need of alpha 1-antitrypsin therapy. In some embodiments, the subject in need of alpha 1-antitrypsin therapy has an alpha 1-antitrypsin deficiency. In some embodiments, a subject in need of alpha 1-antitrypsin therapy is a subject with an inflammatory disease, disorder or condition (e.g., In some afflicted with inflammation). In some embodiments, a subject in need of alpha 1-antitrypsin therapy is a subject with a disease, disorder or condition of the immune system. In some embodiments, a subject in need of alpha 1-antitrypsin therapy is a subject with a disease, disorder or condition characterized by cellular necrosis. In some embodiments, a subject in need of alpha 1-antitrypsin therapy is a subject with a wound.

In some embodiments, a subject in need of alpha 1-antitrypsin therapy requires therapy for a disease, disorder or condition selected from the group consisting of: diabetes, allogenic and xenogeneic transplantation, graft-versus-host disease, myocardial infarction, radiation exposure, chronic fatigue syndrome, bacterial infection, inflammatory bowel disease, rheumatoid arthritis, liver disease, radiation exposure, osteoporosis, multiple sclerosis, neuromyelitis optica, organ injury in patients undergoing cardiac surgery, ischemia-reperfusion associated injuries of the heart and lung, an external wound, an internal wound, skin necrosis, skin damage and osteoporosis.

As used herein, the terms “treatment” or “treating” encompasses any one of: amelioration, prevention, ameliorating, and preventing.

In some embodiments, the inflammatory-associated disease or disorder has an immune system component.

In some embodiments, the disease or disorder with an immune system component is selected from: diabetes, allogenic and xenogeneic transplantation, graft-versus-host disease, bacterial infection, rheumatoid arthritis, inflammatory bowel disease, osteoarthritis, vasculitis, chronic fatigue syndrome and multiple sclerosis.

In some embodiments, the inflammatory-associated disease or disorder has a necrotic component.

In some embodiments, the disease or disorder with a necrotic component is selected from: myocardial infarction, radiation exposure, and liver disease.

In some embodiments, the inflammatory disease or disorder is selected from the group consisting of: diabetes, allogenic and xenogeneic transplantation, graft-versus-host disease, myocardial infarction, radiation exposure, chronic fatigue syndrome, bacterial infection, inflammatory bowel disease, rheumatoid arthritis, liver disease, radiation exposure, osteoporosis, multiple sclerosis, neuromyelitis optica, organ injury in patients undergoing cardiac surgery, ischemia-reperfusion associated injuries of the heart and lung, and osteoporosis.

As used herein, the terms “treatment” or “treating” of a disease, disorder, or condition encompasses alleviation of at least one symptom thereof, a reduction in the severity thereof, or inhibition or slowing of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

As used herein, the term “prevention” of a disease, disorder, or condition encompasses the delay, prevention, suppression, or inhibition of the onset of a disease, disorder, or condition. As used in accordance with the presently described subject matter, the term “prevention” relates to a process of prophylaxis in which a subject is exposed to the presently described polypeptide prior to the induction or onset of the disease/disorder process. This could be done where an individual has a genetic pedigree indicating a predisposition toward occurrence of the disease/disorder to be prevented. For example, this might be true of an individual whose ancestors show a predisposition toward certain types of, for example, inflammatory disorders. The term “suppression” is used to describe a condition wherein the disease/disorder process has already begun but obvious symptoms of the condition have yet to be realized. Thus, the cells of an individual may have the disease/disorder, but no outside signs of the disease/disorder have yet been clinically recognized. In either case, the term prophylaxis can be applied to encompass both prevention and suppression. Conversely, the term “treatment” refers to the clinical application of active agents to combat an already existing condition whose clinical presentation has already been realized in a patient. In some embodiments, treatment refers to a clinical application of active agents to combat an already existing condition whose clinical presentation has yet to be realized in a patient.

As used herein, the term “condition” includes anatomic and physiological deviations from the normal that constitute an impairment of the normal state of the living animal or one of its parts, that interrupts or modifies the performance of the bodily functions.

As used herein, the terms “administering”, “administration”, and like terms refer to any method which, in sound medical practice, delivers a composition containing an active agent to a subject in such a manner as to provide a therapeutic effect. One aspect of the present subject matter provides for dermal or transdermal administration of a therapeutically effective amount of a composition of the present subject matter to a patient in need thereof. Other suitable routes of administration can include oral, dermal, transdermal, parenteral, subcutaneous, intravenous, intramuscular, or intraperitoneal. In some embodiments, the administering is systemic administering. In some embodiments, the administering to the wound. In some embodiments, the administering is to the site of inflammation.

As used herein, the terms “subject” or “individual” or “animal” or “patient” or “mammal,” refers to any subject, particularly a mammalian subject, for whom therapy is desired, for example, a human.

According to some embodiments, there is provided a method for producing a polypeptide characterized by having immune cell modulating activity, comprising: (a) providing a cell comprising the artificial vector of the invention; and culturing the cell of step (a) such that a polypeptide encoded by the artificial is expressed, thereby producing the polypeptide characterized by having immune cell modulating activity.

In some embodiments, the polypeptide is the polypeptide of the invention.

In some embodiments, the method further comprises a step preceding step (a) comprising introducing or transfecting the cell with the artificial vector.

In some embodiments, the method further comprises a step proceeding step (b) comprising isolating, extracting, purifying, or any combination thereof, the polypeptide from the cell, from a medium wherein the cell is cultured, or both.

In some embodiments, there is provided an extract or any portion or fraction thereof, comprising the polypeptide of the invention.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a”, “an”, and “at least one”, are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise,” “include” and “have” and conjugates thereof, are used to indicate that the object or objects of the verb are not necessarily a complete listing of components, elements or parts of the subject or subjects of the verb.

Other terms as used herein are meant to be defined by their well-known meanings in the art.

Unless specifically stated or obvious from context, as used herein, the term “or” is understood to be inclusive.

Throughout this specification and claims, the word “comprise,” or variations such as “comprises” or “comprising,” indicate the inclusion of any recited integer or group of integers but not the exclusion of any other integer or group of integers.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition.

As used herein, the terms “comprises”, “comprising”, “containing”, “having” and the like can mean “includes”, “including”, and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments. In one embodiment, the terms “comprises,” “comprising, “having” are/is interchangeable with “consisting”.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Generally, the nomenclature used herein, and the laboratory procedures utilized in the present invention include chemical, molecular, biochemical, and cell biology techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); “Cell Biology: A Laboratory Handbook”, Volumes I-III Cellis, J. E., ed. (1994); The Organic Chemistry of Biological Pathways by John McMurry and Tadhg Begley (Roberts and Company, 2005); Organic Chemistry of Enzyme-Catalyzed Reactions by Richard Silverman (Academic Press, 2002); Organic Chemistry (6^{th} Edition) by Leroy “Skip” G Wade; Organic Chemistry by T. W. Graham Solomons and, Craig Fryhle.

### Materials and Methods

### Plasmid Constructs

Plasmid constructs were based on a pFUSE plasmid (Open Biosystems, GE Healthcare, Chicago, Ill., USA) containing WT human AAT (hAAT) clone with a His-tag sequence at the C-terminal (PMID 29780379). Plasmids were replicated in DH-5a E. coli (Bio-Lab, Jerusalem, Israel) and purified using Presto Mini Plasmid Kit (Geneaid Biotech, New Taipei City, Taiwan), according to manufacturer's instructions.

hAAT Library Generation

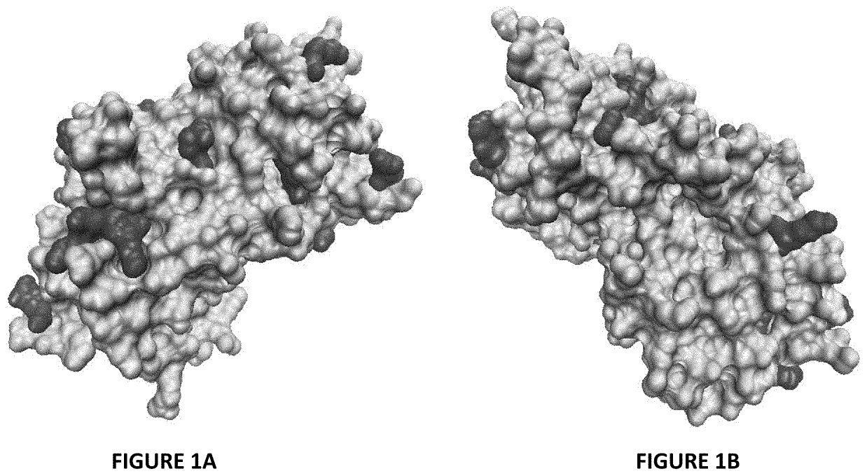

The genetic sequence of hAAT was aligned with 14 mammalian AAT orthologs (Table 1) using Biomatters Geneious program (Auckland, New Zealand) allowing the identification of 35 ‘evolutionary flexible’ sites on the surface of hAAT (FIG. 1).

TABLE 1
AAT orthologs used for sequence alignment

| # | Species (Accession number) |
|---|---|
| 1. | Homo sapiens (AAB59495) |
| 2. | Pan paniscus (XP_003832864) |
| 3. | Sus scrofa (NP_999560) |
| 4. | Bos taurus (CAA44840) |
| 5. | Ovis aries (CAA33561) |
| 6. | Orcinus orca (XP_004262415) |
| 7. | Equus caballus (AAC83412) |
| 8. | Odobenus rosmarus divergens (XP_004394562) |
| 9. | Felis catus (XP_006933139.1) |
| 10. | Pteropus alecto (ELK01169) |
| 11. | Myotis davidii (ELK27058) |
| 12. | Tupaia chinensis (ELV12135) |
| 13. | Rattus norvegicus (AAA40788) |
| 14. | Oryctolagus cuniculus (AAA57133) |

hAAT mutant library was designed using back-to-consensus approach in the ISOR (incorporation of synthetic nucleotide via gene reassembly) methodology (PMID 17483523). The human AAT gene (SERPINA1) was amplified by PCR and ˜10 μg were digested with DNase I (Sigma Aldrich) to yield 50-125 bp fragments, as previously described (PMID 7938023). The fragments were reassembled, as in DNA shuffling (PMID 14695884), in the presence of a mixture of 35 short oligonucleotides (4-6 nM each, Table 2), resulting in 3 libraries holding a total of 103 variants with 2-7 mutations in each gene (PMID 7938023, 14695884). The reaction mix was further amplified by nested PCR as described (PMID 17406305). The assembled libraries were ligated into the pFUSE plasmid, amplified in DH-5a E. coli and purified using Presto Mini Plasmid Kit. A total of 3 back-to-consensus libraries.

TABLE 2
Specific mutation loci and primer list

| # | Mutation | Forward primer sequence (5′-3′) |
|---|---|---|
| 1. | K10E | GGAGATGCTGCCCAGGAGACAGATACATCCCAC (SEQ ID |
| | | NO: 24) |
| 2. | T22A | ATCAGGATCACCCAGCCTTCAACAAGATCACCC (SEQ ID |
| | | NO: 25) |
| 3. | F23C | CAGGATCACCCAACCTGCAACAAGATCACCCC (SEQ ID |
| | | NO: 26) |
| 4. | N24H | GATCACCCAACCTTCCACAAGATCACCCCCAAC (SEQ ID |
| | | NO: 27) |
| 5. | T27A | ACCTTCAACAAGATCGCCCCCAACCTGGCTGAG (SEQ ID |
| | | NO: 28) |
| 6. | E32D | CCCCAACCTGGCTGATTTCGCCTTCAGCCTAT (SEQ ID NO: |
| | | 29) |
| 7. | L41V | GCCTATACCGCCAGGTGGCACACCAGTCCAACA (SEQ ID |
| | | NO: 30) |
| 8. | S47T | ACACCAGTCCAACACCACCAATATCTTCTTCTC (SEQ ID |
| | | NO: 31) |
| 9. | T68A | GCTCTCCCTGGGGGCCAAGGCTGACACTCACG (SEQ ID |
| | | NO: 32) |
| 10. | A70G | CCCTGGGGACCAAGGGTGACACTCACGATGAA (SEQ ID |
| | | NO: 33) |
| 11. | D74T | AAGGCTGACACTCACACTGAAATCCTGGAGGGC (SEQ ID |
| | | NO: 34) |
| 12. | E75Q | CTGACACTCACGATCAAATCCTGGAGGGCCTG (SEQ ID |
| | | NO: 35) |
| 13. | P88A | ACCTCACGGAGATTGCGGAGGCTCAGATCCATG (SEQ ID |
| | | NO: 36) |
| 14. | L120I | GCAATGGCCTGTTCATCAGCGAGGGCCTGAA (SEQ ID NO: |
| | | 37) |
| 15. | S121N | ATGGCCTGTTCCTCAACGAGGGCCTGAAGCTAG (SEQ ID |
| | | NO: 38) |
| 16. | G123S | CTGTTCCTCAGCGAGAGCCTGAAGCTAGTGG (SEQ ID NO: |
| | | 39) |
| 17. | K136N | GCAATGGCCTGTTCATCAGCGAGGGCCTGAA (SEQ ID NO: |
| | | 40) |
| 18. | K201E | GACCCTTTGAAGTCGAGGACACCGAGGAAGAG (SEQ ID |
| | | NO: 41) |
| 19. | D202H | CCTTTGAAGTCAAGCACACCGAGGAAGAGGAC (SEQ ID |
| | | NO: 42) |
| 20. | E204T | GAAGTCAAGGACACCACGGAAGAGGACTTCCAC (SEQ ID |
| | | NO: 43) |
| 21. | Q212E | GACTTCCACGTGGACGAGGTGACCACCGTGAAG (SEQ ID |
| | | NO: 44) |
| 22. | N228D | CGTTTAGGCATGTTTGACATCCAGCACTGTAAG (SEQ ID |
| | | NO: 45) |
| 23. | Q230H | GGCATGTTTAACATCCACCACTGTAAGAAGCTG (SEQ ID |
| | | NO: 46) |
| 24. | K243D | TGGGTGCTGCTGATGGACTACCTGGGCAATGCC (SEQ ID |
| | | NO: 47) |
| 25. | L245V | CTGCTGATGAAATACGTGGGCAATGCCACCGC (SEQ ID |
| | | NO: 48) |
| 26. | V264E | GAAACTACAGCACCTGGAAAATGAACTCACCC (SEQ ID |
| | | NO: 49) |
| 27. | I272L | ACTCACCCACGATATCTTAACCAAGTTCCTGGA (SEQ ID |
| | | NO: 50) |
| 28. | T294S | CCAAACTGTCCATTAGTGGAACCTATGATCTGA (SEQ ID |
| | | NO: 51) |
| 29. | V323I | GCTGACCTCTCCGGGATCACAGAGGAGGCAC (SEQ ID NO: |
| | | 52) |
| 30. | M353T | GAAGCTGCTGGGGCCACGTTTTTAGAGGCCATAC (SEQ ID |
| | | NO: 53) |
| 31. | K370R | GAGGTCAAGTTCAACCGACCCTTTGTCTTCTTA (SEQ ID |
| | | NO: 54) |
| 32. | M376I | CCCTTTGTCTTCTTAATCATTGAACAAAATAC (SEQ ID NO: |
| | | 55) |
| 33. | I377Y | CTTTGTCTTCTTAATGTACGAACAAAATACCAA (SEQ ID |
| | | NO: 56) |
| 34. | E378D | GTCTTCTTAATGATTGACCAAAATACCAAGTC (SEQ ID NO: |
| | | 57) |
| 35. | M387V | AAGTCTCCCCTCTTCGTGGGAAAAGTGGTGAATC (SEQ ID |
| | | NO: 58) |

Single mutation variants

Single mutations were generated by back-to-back amplification using Q5 high-fidelity DNA polymerase (New England Biolabs, Ipswich, Mass., USA) as described (PMID 19566935). The following primers were used.

TABLE 3
Primers used in back-to-back mutagenesis for site-specific mutagenesis.

| Mutation | Forward (5′-3′) | Reverse (5′-3′) |
|---|---|---|
| T22A | CACCCAgCCTTCAACAAGATC | GAAGGcTGGGTGATCCTGAT |
| | ACCCCCAAC (SEQ ID NO: 10) | CATGGTGGG (SEQ ID NO: 11) |
| T68A | CTGGGGgCCAAGGCTGACAC | CTTGGcCCCCAGGGAGAGCA |
| | TCAC (SEQ ID NO: 12) | TTGC (SEQ ID NO: 13) |
| D74T | CACTCACacTGAAATCCTGGA | GATTTCAgtGTGAGTGTCAGC |
| | GGGCCTG (SEQ ID NO: 14) | CTTGGTCCCC (SEQ ID NO: |
| | | 15) |
| D74T | — | GATTTCAgtGTGAGTGTCAGC |
| and | | CTTGGcCCCC (SEQ ID NO: |
| T68A | | 16) |
| D202H | GTCAAGcACACCGAGGAAGA | GGTGTgCTTGACTTCAAAGG |
| | GGACTTCCAC (SEQ ID NO: 17) | GTCTCTCCCATTTG (SEQ ID |
| | | NO: 18) |
| K243D | GCTGATGgAtTACCTGGGCAA | CCAGGTAaTcCATCAGCAGC |
| | TGCCACCG (SEQ ID NO: 19) | ACCCAGCTGG (SEQ ID NO: |
| | | 20) |
| K368R | CAACAgACCCTTTGTCTTCTT | AGGGTcTGTTGAACTTGACC |
| | AATGATTGAACAAAATACCA | TCGGGG (SEQ ID NO: 22) |
| | AGTCTCC (SEQ ID NO: 21) | |

In Table 3, capital letters represent polynucleotide sequences which are identical to the wild type hAAT, whereas lower case letters indicate the specific mutations which were introduced into the expressed transcripts so as to obtain the mhAAT variants disclosed herein.

### Recombinant Protein Production and Purification

Gene library was expressed in HEK-293T cells (CRL-3216, ATCC, Manassas, W. Va., USA). Cells were cultured in complete DMEM (containing 10% fetal bovine serum, 50 U/ml streptomycin/penicillin, 50 μg/ml L-glutamine, all from Biological Industries) in a 5% CO₂ humidified incubator, and transiently transfected using GeneTranTM transfection reagent (Biomega, San Diego, Calif., USA) according to manufacturer's instructions. Six days post-transfection, supernatants were collected, hAAT content was assessed by species specific ELISA (ICL, Portland, Oreg., USA) and anti-inflammatory screen initiated without protein purification.

WT-rhAAT, MJ6-rhAAT and specific single mutation variants were transfected using polyethyleneimine (PEI, Sigma Aldrich) as described (PMID 24011049). Six days post-transfection, supernatants were collected and secreted rhAAT purified using Ni beads (Calbiochem, Merck Millipore, Darmstadt, Germany) by standard protocol. Following protein purification, samples were treated with dialysis (SnakeSkin dialysis tubing, 3.5 K MWCO, ThermoFisher Scientific) against PBS (Biological industries) to eliminate the presence of imidazole.

After purification, samples were assessed for purity and molecular weight by Ponceau S stain (Sigma Aldrich) and western blotting using an anti-human Alpha1-antitrspin antibody (Sigma Aldrich), after electrophoresis on a 10% polyacrylamide gel. Commercial clinical-grade serum-purified hAAT (Glassia, Kamada, Ness-Ziona, Israel) was used as reference (data not shown). Protein concentrations were determined using micro-volume spectrophotometer (Nanodrop, ThemoFisher Scientific, Waltham, Mass., USA) and Bradford Protein Assay (Bio-Rad Laboratories, Rishon-LeZion, Israel).

### Mice

Six to eightweeks old males and females C57BL/6 mice (Harlan Laboratories Ltd., Jerusalem, Israel) were used for all the experiments, with Ben-Gurion University of the Negev Animal Care and Use Committee approval.

### Bone Marrow-Derived Macrophages (BMDM)

The tibia and femur of C57BL/6 mice were surgically removed and thoroughly washed with 4° C. PBS (Biological Industries) through a 70- M sterile nylon cell strainer (BD; Becton Dickinson and company, Franklin Lakes, N.J., USA). The remaining cells were cultured in 10 ml of complete RPMI-1640 (containing 10% fetal bovine serum, 50 U/ml streptomycin/penicillin, 50 μg/ml L-glutamine, all from Biological Industries), 50 μM 2-ME (Sigma-Aldrich) and 20 ng/ml recombinant Granulocyte Macrophage Colony-Stimulating Factor (rGM-CSF, PeproTech, Rocky Hill, N.J., USA). On days 3 and 6 medium containing rGM-CSF was added. Cell populations were confirmed as being >95% CD11b⁺ after 9 days of incubation with rGM-CSF by flow cytometry.

### Thioglycolate-Elicited Primary Peritoneal Cell

C57BL/6 mice were intraperitoneally injected (i.p) with thioglycolate (3% (v/v), Sigma-Aldrich; i.p, 1.5 ml per mouse). Five days later, peritoneal lavage was performed with cold PBS (biological industries) and recovered liquid filtered through 70-μM sterile nylon strainer. Cells were then centrifuged resuspended in complete RPMI 1640. Cell cultures were routinely verified to be >95% CD11b⁺/F4-80⁺ cells by flow cytometry.

### Cell Activation Assays and Flow Cytometry

BMDMs and peritoneal macrophages were seeded (3×10⁵ cells per well in 300 μl complete RPMI 1640, in triplicates) and cultured overnight with or without rhAAT at indicated concentrations, in standard conditions (37° C., 5% CO2 humidified incubator).

Wells were then gently washed with PBS and medium replaced with the same concentrations of rhAAT variants, as well as LPS (Sigma-Aldrich) to a final concentration of 5 ng/ml. Twenty-four hours later, supernatants were collected and analyzed for IL-6 concentrations using specific ELISA (Biolegend, San Diego, Calif., USA). Cells were gently removed with a designated policeman and suspended in FACS buffer (PBS containing 1% BSA from Biological Industries, 0.1% sodium azide and 2 mM EDTA, both from Sigma-Aldrich). CD16/32 blocking was achieved by room temperature 20 minutes incubation with anti-CD16/32 antibody (Biolegend). F4-80, CD40 and CD86 staining was achieved by an additional 20-minutes incubation at 4° C. with anti-mouse antibodies: anti-CD40-FITC (3/2.3), anti-CD86-PE (GL-1) all from Biolegend, and anti-F4-80-PerCP-Cy5.5 (BM8.1) (Mere, Temecula, Calif., USA). Fluorescent readout was determined in BD Canto II and data analyzed by FLOWJO 10.0.8r1 software (Flowjo, LLC Data Analysis Software, Af shland, Oregon, USA). After exclusion of cellular debris and duplicated cells, F4-80+ population was selected and surface expression levels of CD40 and CD86 were assessed and compared between samples.

### Neutrophil Elastase Activity Assay

Neutrophil elastase inhibitory potency was determined in acellular conditions using a designated kit (R&D Systems Sigma-Aldrich, Lois, MO, Minneapolis, Minn., USA), according to manufacturer's instruction (final elastase concentration per well: 0.39 M). rhAAT variants were pre-incubated with the commercial enzyme for 10 minutes in 37° C. prior to kinetic evaluation of color-producing substrate processing.

### Statistical Analysis

Two-tailed Mann-Whitney test was used to assess differences between selected experimental conditions. Results are expressed as mean±SEM, p<0.05 was considered significant. All statistical analyses were performed using GraphPad Prism version 7.

### Example 1

### AAT Library Generation and Expression

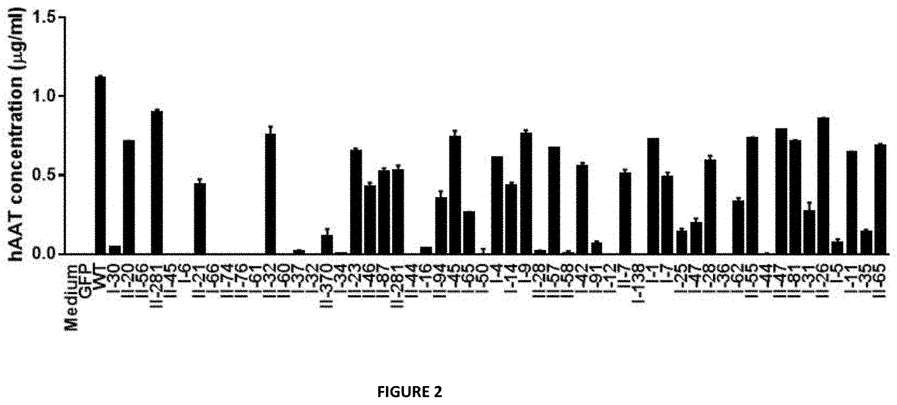

hAAT gene library screen was conducted by variants transient transfection to HEK-293F cells, followed by AAT concentrations quantification in 6-day supernatants (FIG. 2). WT-rhAAT was readily expressed (1.11 μg/ml). Variants expression was found to vary and ranged between expression levels similar to that of WT-rhAAT (e.g., II-281, I-26 with 0.9 μg/ml and 0.85 μg/ml, respectively), reduced expression (e.g., I-91, II-370 with 0.07 μg/ml and 0.11 g/ml, respectively) to no detected expression (e.g., II-74, II-76 with 0 μg/ml). Of the 103 variants in the libraries, 48 (46.6%) variants were expressed in satisfactory quantities, and thus included in the test.

### Example 2

### Anti-Inflammatory Variation of Expressed Variants

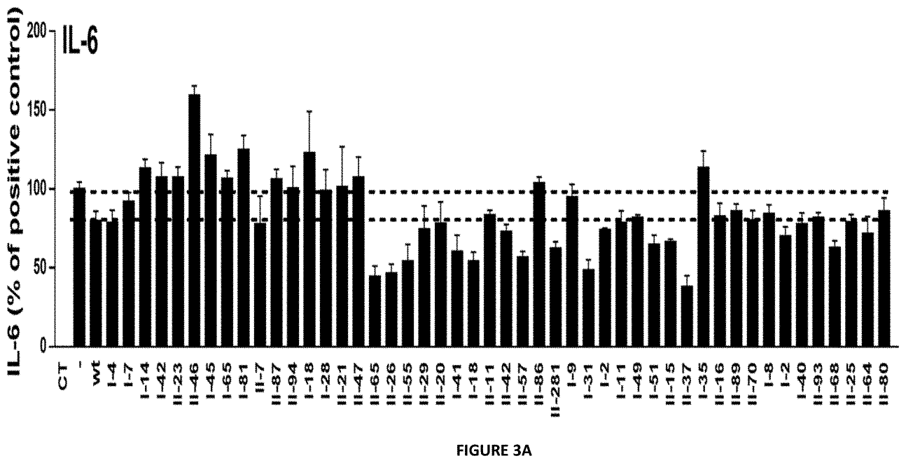

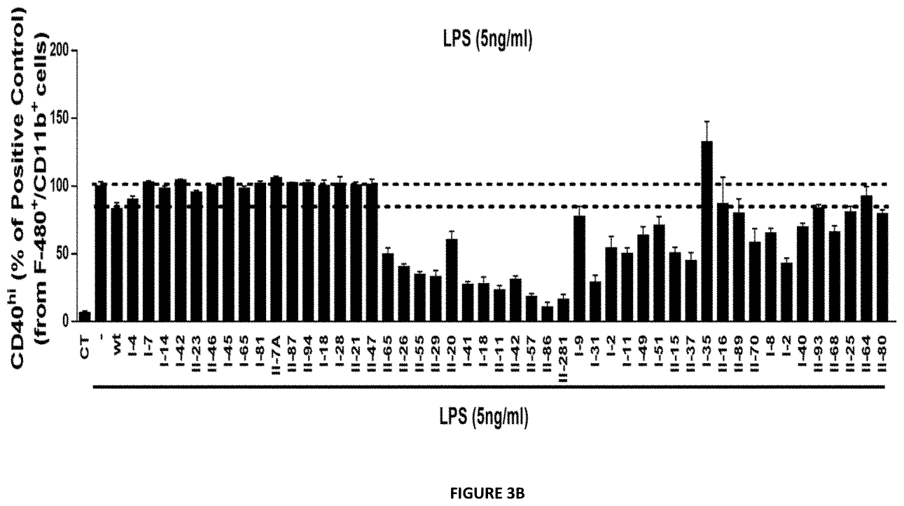

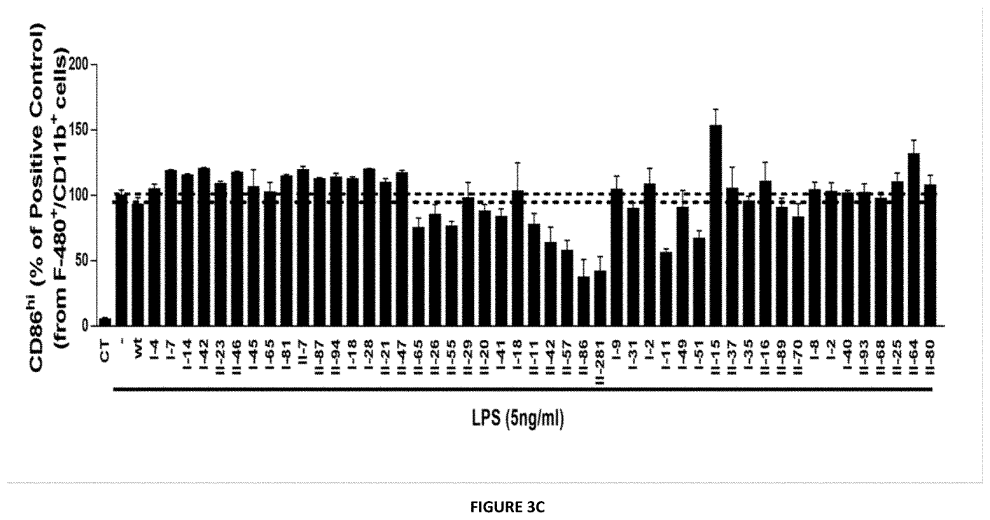

The anti-inflammatory potency of selected variants was assessed in LPS-stimulated primary murine BMDM cells (FIG. 3). The inventors have shown that cellular response to LPS included a marked increase in inducible IL-6 supernatant quantities (FIG. 3A) as well as an increase of surface expression of CD40 and CD86 (FIGS. 3B-3C). Exposure to supernatant of WT-rhAAT transfected cells resulted in a 20% reduction in inducible IL-6 levels as well as a 17% and 7% reduction in CD40 and CD86 surface expression, respectively. Pretreatment with other variants resulted in marked variations in cellular response varying from increased inflammatory response (e.g., II-46 with a 60%, 0% and 17% increase in IL-6, CD40 and CD86 expression, respectively), through neutral effect (e.g., I-65 with a 7% increase in IL-6 secretion and 0% increase in CD40 and CD86 expression, respectively), to reduced inflammatory response (e.g., 11-281 with a 38%, 84% and 58% reduction in IL-6, CD40 and CD86 expression, respectively, FIG. 3).

### Example 3

### Mutation Selection for Reintegration

A threshold of 20% reduction in IL-6, CD40 and CD86 compared with non-treated cells was defined as the criteria for positive selection. Only 9 variants (II-65, II-55, II-11, II-42, II-57, II-86, II-281, I-11, I-51) met the aforementioned criteria and therefore were considered eligible for inclusion in this phase of the study and underwent further sequencing (Table 4).

TABLE 4
List of mutation in nine selected AAT variants

| Variant | Mutations | | | | | | | # of mutations |
|---|---|---|---|---|---|---|---|---|
| 11-65 | F23C | S121N | K201E | N228D | L245V | K368R | | 6 |
| 11-55 | T68A | D74T | Q230H | N243D | K368R | E376D | | 6 |
| 11-11 | T22A | T68A | D74T | D202H | G225R | K243D | | 6 |
| 11-42 | T22A | N46S | M63I | D202H | Q230H | K243D | K368R | 7 |
| 11-57 | N24H | K136N | D202H | K243D | E376D | | | 5 |
| 11-86 | N24H | E32D | T68A | D74T | Q212E | | | 5 |
| 11-281 | T22A | K243D | K368R | E376D | | | | 4 |
| 1-11 | N24H | T68A | S121N | K243D | K368R | | | 5 |
| 1-51 | T22A | D74T | L120I | D202H | K136N | K368R | | 6 |

Mutations selection of reintegration into WT-rhAAT was performed base on mutation frequency in the examined cohort. Mutations frequency analysis identified 6 mutations (T22A, T68A, D74T, D202H, K243D and K368R) as the most common mutations in the examined cohort, with a minimal frequency of 4 variants.

### Example 4

### The MJ6 Variant

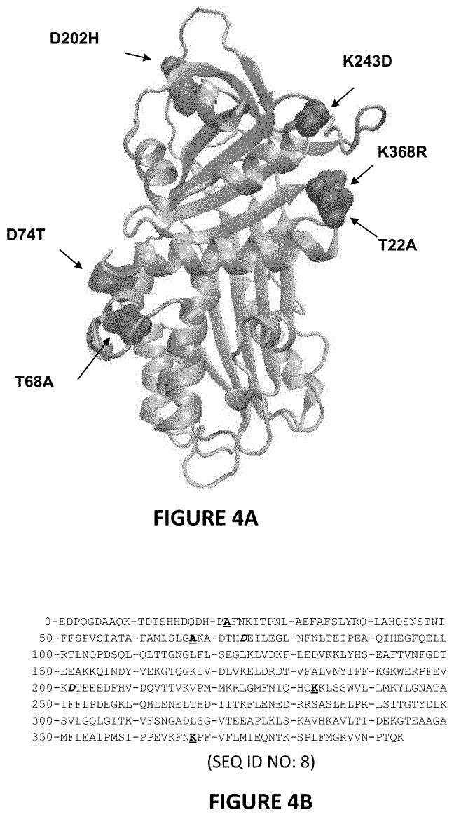

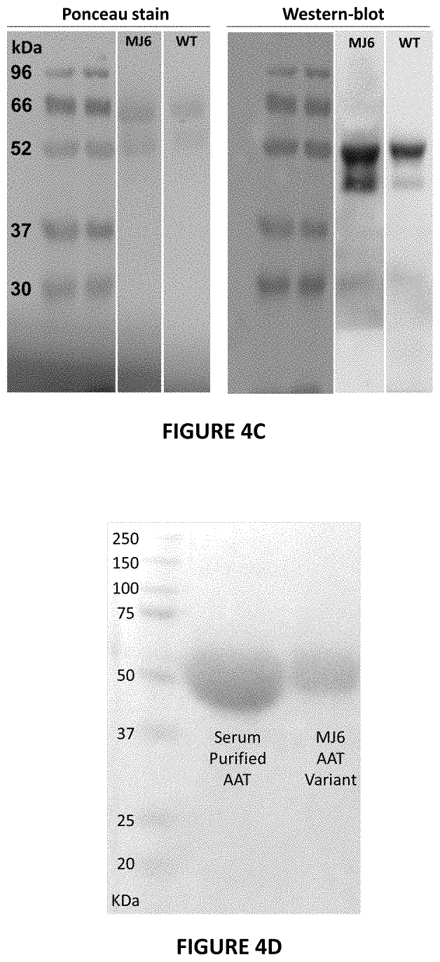

Six selected point mutations were integrated into wt-rhAAT sequence in a pFUSE plasmid to form the sequence of MJ6-rhAAT (FIGS. 4A-4B). HEK293T cells were then transiently transfected with the wt-rhAAT or MJ6-rhAAT plasmid and allowed to release His-tagged proteins into the supernatant for 6 days. Proteins were purified by affinity chromatography and size and purity were assessed using polyacrylamide gel electrophoresis (PAGE), Ponceau S stain, and western blotting (FIGS. 4C-4D). Ponceau S stain has identified two bands at ˜52 kDa and ˜66 kDa, while western blotting identified bands at ˜52 kDa and ˜44 kDa (FIG. 4C).

### Example 5

### Elastase Inhibitory Activity

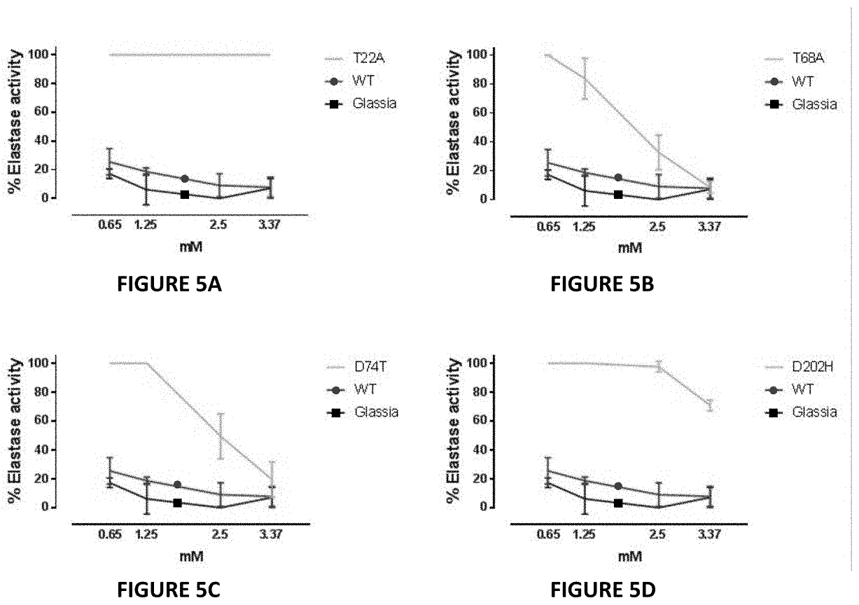

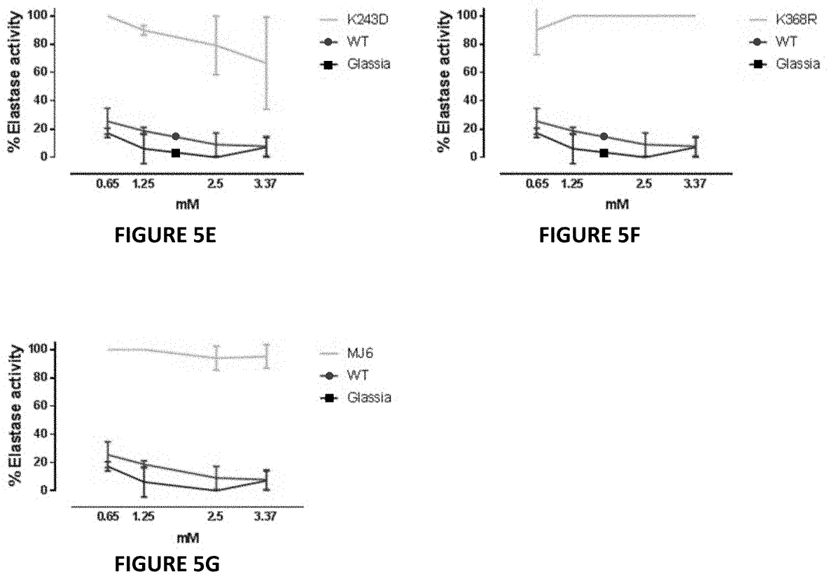

Elastase inhibitory activity was assessed in vitro by incubating commercial recombinant neutrophil elastase (NE) with WT-rhAAT, MJ6-rhAAT or other variants harboring a single mutation. WT-rhAAT incubation with NE in concentrations as low as 0.65 μM resulted in a profound 75% reduction of NE activity (FIGS. 5A-5G), while incubation with MJ6-rhAAT did not affect the proteolytic activity of NE in any of the tested concentrations (FIG. 5G).

Variants examination revealed that T22A and K368R did not inhibit neutrophil elastase in any of the tested concentrations (FIG. 5A and FIG. 5F, respectively). Variants comprising T68A or D74T were able to achieve an inhibitory potency similar to WT-rhAAT when applied in a concentration of 3.37 μM (FIG. 5B and FIG. 5C, respectively). Variants comprising D202H or K243 displayed an inhibitory activity, somewhat lower than the WT-rhAAT, in any of the tested concentrations (FIG. 5D and FIG. 5E, respectively).

### Example 6

### Anti-Inflammatory Activity

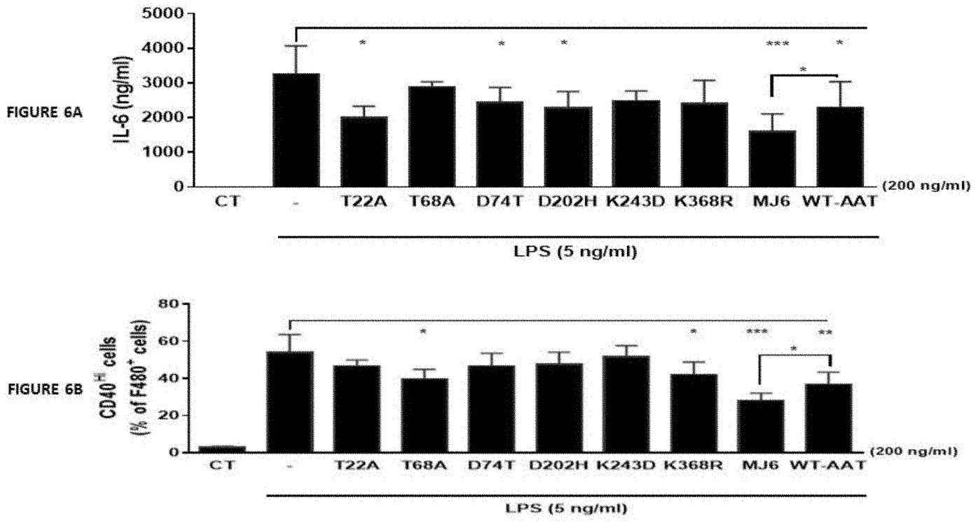

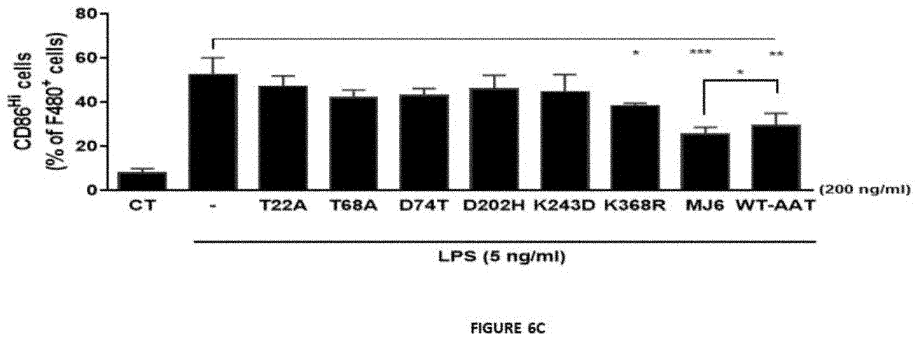

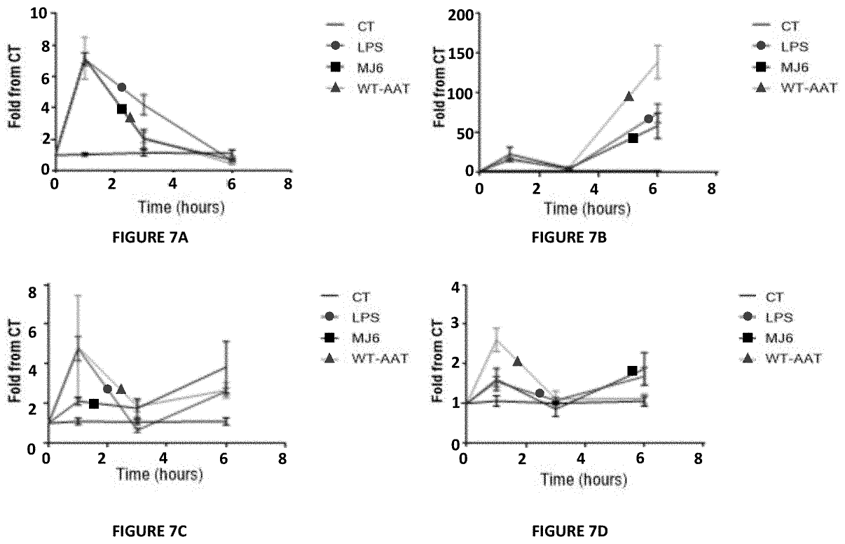

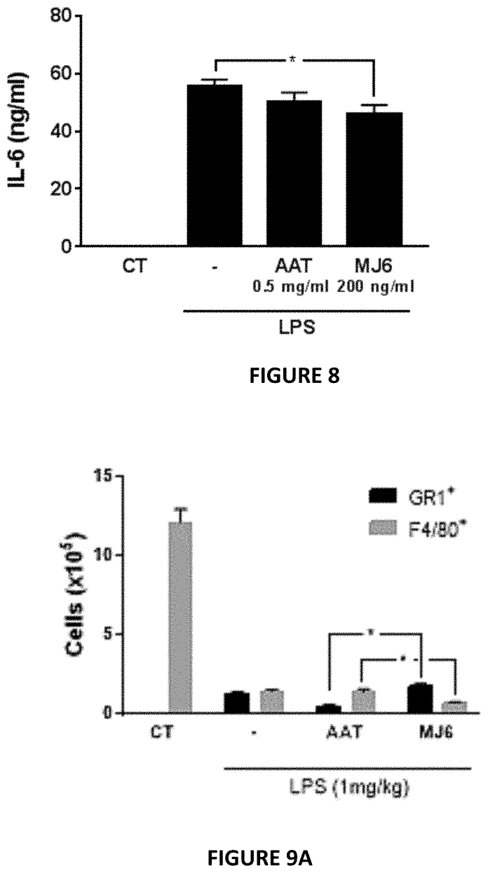

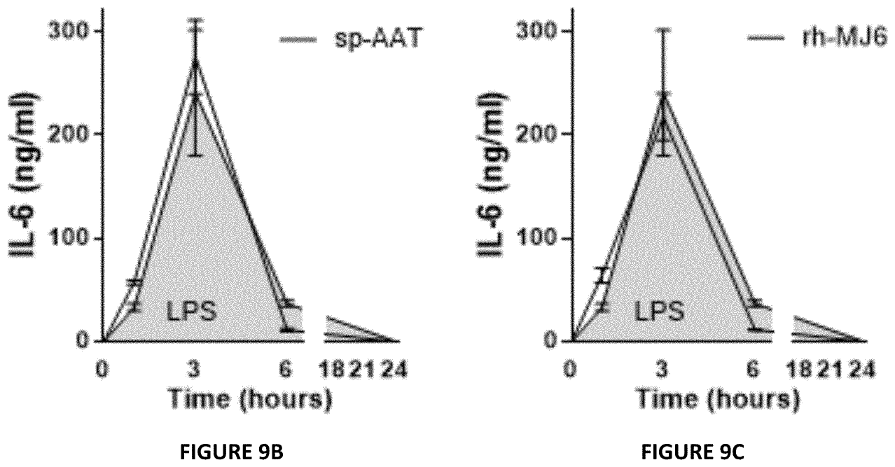

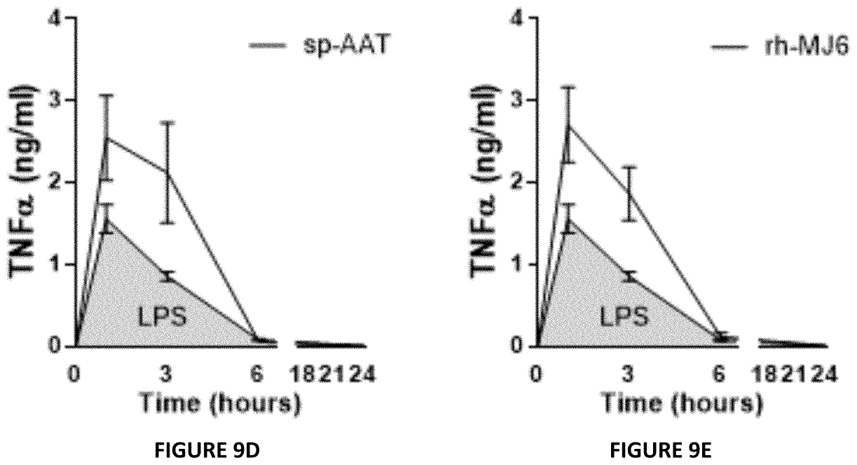

LPS-stimulatory response of primary murine peritoneal cells was tested in the presence of WT-rhAAT, MJ6-rhAAT or variants harboring a single mutation (FIG. 6). The inventors have shown that LPS stimulation of primary murine peritoneal cells resulted in an increase in supernatant IL-6 (FIG. 6A) and surface expression of CD40 and CD86 (FIGS. 6B-6C, respectively). WT-rhAAT pretreatment resulted in a significant reduction of inducible IL-6, CD40 and CD86 (29.6% p<0.05, 31.9% p<0.01 and 44% p<0.01 compared with LPS alone, respectively) as did pretreatment with MJ6-rhAAT (50.8% p<0.001, 47.7% p<0.001 and 51.6% p<0.001 compared with LPS alone, respectively). Upon comparison between WT-rhAAT and MJ6-rhAAT, a statistically significant superior anti-inflammatory effect was observed for MJ6-rhAAT in terms of inducible IL-6, CD40 and CD86 reduction (30% p<0.05, 23% p<0.05, 14% p<0.05, compared with WT-rhAAT pretreatment, respectively). Pretreatment with other mutant variants carrying a single mutation varied in potency. While pretreatment with mutant variants of hAAT comprising T22A, D74T, D202H or K243D resulted in a significant reduction of supernatant IL-6 content, lower changes in CD40 and CD86 expression were noted, compared to the MJ6 variant. Pretreatment with mutant variants of hAAT comprising T68A or K368R resulted in a significant reduction of CD40 and CD86 surface expression accompanied by a reduction of inducible IL-6 levels (this reduction was not statistically supported). Reduction of the aforementioned assessed outcomes following pretreatment with a variant mutant of hAAT comprising K243D were observed but was not statistically supported.

While certain features of the invention have been described herein, many modifications, substitutions, changes, and equivalents will now occur to those of ordinary skill in the art. It is, therefore, to be understood that the appended claims are intended to cover all such modifications and changes as fall within the true spirit of the invention.

1. A polypeptide comprising the amino acid sequence of EDPQGDAAQKTDTSHHDQDHPTFNKITPNLAEFAFSLYRQLAHQSNSTNIFFSPVSIATAF AMLSLGTKADTHDEILEGLNFNLTEIPEAQIHEGFQELLHTLNQPDSQLQLTTGNGLFLSE GLKLVDKFLEDVKKLYHSEAFTVNFGDTEEAKKQINDYVEKGTQGKIVDLVKELDRDT VFALVNYIFFKGKWERPFEVKDTEEEDFHVDQVTTVKVPMMKRLGMFNIQHCKKLSS WVLLMKYLGNATAIFFLPDEGKLQHLENELTHDIITKFLENEDRRSASLHLPKLSITGTY DLKSILGQLGITKVFSNGADLSGVTEEAPLKLSKAVHKAVLTIDEKGTEAAGAMFLEAIP MSIPPEVKFNKPFVFLMIEQNTKSPLFMGKVVNPTQK (SEQ ID NO: 9), wherein said polypeptide comprises at least one amino acid substitution at a position selected from the group consisting of: T22, T68, D74, D202, K243, and K368.

2. The polypeptide of claim 1, comprising the amino acid sequence of EDPQGDAAQKTDTSHHDQDHPX₁FNKITPNLAEFAFSLYRQLAHQSNSTNIFFSPVSIATA FAMLSLGX₂KADTHX₃EILEGLNFNLTEIPEAQIHEGFQELLHTLNQPDSQLQLTTGNGLF LSEGLKLVDKFLEDVKKLYHSEAFTVNFGDTEEAKKQINDYVEKGTQGKIVDLVKELD RDTVFALVNYIFFKGKWERPFEVKX₄TEEEDFHVDQVTTVKVPMMKRLGMFNIQHCKK LSSWVLLMX₅YLGNATAIFFLPDEGKLQHLENELTHDIITKFLENEDRRSASLHLPKLSIT GTYDLKSILGQLGITKVFSNGADLSGVTEEAPLKLSKAVHKAVLTIDEKGTEAAGAMFL EAIPMSIPPEVKFNX₆PFVFLMIEQNTKSPLFMGKVVNPTQK (SEQ ID NO: 1), wherein X₁ is T or A; X₂ is T or A; X₃ is D or T; X₄ is D or H; X₅ is K or D; and X₆ is K or R.

3. The polypeptide of claim 1, comprising the amino acid sequence of any one of:
(SEQ ID NO: 2)
a.
EDPQGDAAQKTDTSHHDQDHPAFNKITPNLAEFAFSLYRQLAHQSNSTNIFFSPVSIA
TAFAMLSLGTKADTHDEILEGLNFNLTEIPEAQIHEGFQELLHTLNQPDSQLQLTTGN
GLFLSEGLKLVDKFLEDVKKLYHSEAFTVNFGDTEEAKKQINDYVEKGTQGKIVDL
VKELDRDTVFALVNYIFFKGKWERPFEVKDTEEEDFHVDQVTTVKVPMMKRLGMF
NIQHCKKLSSWVLLMKYLGNATAIFFLPDEGKLQHLENELTHDIITKFLENEDRRSAS
LHLPKLSITGTYDLKSILGQLGITKVFSNGADLSGVTEEAPLKLSKAVHKAVLTIDEK
GTEAAGAMFLEAIPMSIPPEVKFNKPFVFLMIEQNTKSPLFMGKVVNPTQK; [[,]]
(SEQ ID NO: 3)
b.
EDPQGDAAQKTDTSHHDQDHPTFNKITPNLAEFAFSLYRQLAHQSNSTNIFFSPVSIA
TAFAMLSLGAKADTHDEILEGLNFNLTEIPEAQIHEGFQELLHTLNQPDSQLQLTTGN
GLFLSEGLKLVDKFLEDVKKLYHSEAFTVNFGDTEEAKKQINDYVEKGTQGKIVDL
VKELDRDTVFALVNYIFFKGKWERPFEVKDTEEEDFHVDQVTTVKVPMMKRLGMF
NIQHCKKLSSWVLLMKYLGNATAIFFLPDEGKLQHLENELTHDIITKFLENEDRRSAS
LHLPKLSITGTYDLKSILGQLGITKVFSNGADLSGVTEEAPLKLSKAVHKAVLTIDEK
GTEAAGAMFLEAIPMSIPPEVKFNKPFVFLMIEQNTKSPLFMGKVVNPTQK;
c.
(SEQ ID NO: 4)
EDPQGDAAQKTDTSHHDQDHPTFNKITPNLAEFAFSLYRQLAHQSNSTNIFFSPVSIA
TAFAMLSLGTKADTHTEILEGLNFNLTEIPEAQIHEGFQELLHTLNQPDSQLQLTTGN
GLFLSEGLKLVDKFLEDVKKLYHSEAFTVNFGDTEEAKKQINDYVEKGTQGKIVDL
VKELDRDTVFALVNYIFFKGKWERPFEVKDTEEEDFHVDQVTTVKVPMMKRLGMF
NIQHCKKLSSWVLLMKYLGNATAIFFLPDEGKLQHLENELTHDIITKFLENEDRRSAS
LHLPKLSITGTYDLKSILGQLGITKVFSNGADLSGVTEEAPLKLSKAVHKAVLTIDEK
GTEAAGAMFLEAIPMSIPPEVKFNKPFVFLMIEQNTKSPLFMGKVVNPTQK;
d.
(SEQ ID NO: 5)
EDPQGDAAQKTDTSHHDQDHPTFNKITPNLAEFAFSLYRQLAHQSNSTNIFFSPVSIA
TAFAMLSLGTKADTHDEILEGLNFNLTEIPEAQIHEGFQELLHTLNQPDSQLQLTTGN
GLFLSEGLKLVDKFLEDVKKLYHSEAFTVNFGDTEEAKKQINDYVEKGTQGKIVDL
VKELDRDTVFALVNYIFFKGKWERPFEVKHTEEEDFHVDQVTTVKVPMMKRLGMF
NIQHCKKLSSWVLLMKYLGNATAIFFLPDEGKLQHLENELTHDIITKFLENEDRRSAS
LHLPKLSITGTYDLKSILGQLGITKVFSNGADLSGVTEEAPLKLSKAVHKAVLTIDEK
GTEAAGAMFLEAIPMSIPPEVKFNKPFVFLMIEQNTKSPLFMGKVVNPTQK;
e.
(SEQ ID NO: 6)
EDPQGDAAQKTDTSHHDQDHPTFNKITPNLAEFAFSLYRQLAHQSNSTNIFFSPVSIA
TAFAMLSLGTKADTHDEILEGLNFNLTEIPEAQIHEGFQELLHTLNQPDSQLQLTTGN
GLFLSEGLKLVDKFLEDVKKLYHSEAFTVNFGDTEEAKKQINDYVEKGTQGKIVDL
VKELDRDTVFALVNYIFFKGKWERPFEVKDTEEEDFHVDQVTTVKVPMMKRLGMF
NIQHCKKLSSWVLLMDYLGNATAIFFLPDEGKLQHLENELTHDIITKFLENEDRRSAS
LHLPKLSITGTYDLKSILGQLGITKVFSNGADLSGVTEEAPLKLSKAVHKAVLTIDEK
GTEAAGAMFLEAIPMSIPPEVKFNKPFVFLMIEQNTKSPLFMGKVVNPTQK;
f.
(SEQ ID NO: 7)
EDPQGDAAQKTDTSHHDQDHPTFNKITPNLAEFAFSLYRQLAHQSNSTNIFFSPVSIA
TAFAMLSLGTKADTHDEILEGLNFNLTEIPEAQIHEGFQELLHTLNQPDSQLQLTTGN
GLFLSEGLKLVDKFLEDVKKLYHSEAFTVNFGDTEEAKKQINDYVEKGTQGKIVDL
VKELDRDTVFALVNYIFFKGKWERPFEVKDTEEEDFHVDQVTTVKVPMMKRLGMF
NIQHCKKLSSWVLLMKYLGNATAIFFLPDEGKLQHLENELTHDIITKFLENEDRRSAS
LHLPKLSITGTYDLKSILGQLGITKVFSNGADLSGVTEEAPLKLSKAVHKAVLTIDEK
GTEAAGAMFLEAIPMSIPPEVKFNRPFVFLMIEQNTKSPLFMGKVVNPTQK;
and
g.
(SEQ ID NO: 8)
EDPQGDAAQKTDTSHHDQDHPAFNKITPNLAEFAFSLYRQLAHQSNSTNIFFSPVSIA
TAFAMLSLGAKADTHTEILEGLNFNLTEIPEAQIHEGFQELLHTLNQPDSQLQLTTGN
GLFLSEGLKLVDKFLEDVKKLYHSEAFTVNFGDTEEAKKQINDYVEKGTQGKIVDL
VKELDRDTVFALVNYIFFKGKWERPFEVKHTEEEDFHVDQVTTVKVPMMKRLGMF
NIQHCKKLSSWVLLMDYLGNATAIFFLPDEGKLQHLENELTHDIITKFLENEDRRSAS
LHLPKLSITGTYDLKSILGQLGITKVFSNGADLSGVTEEAPLKLSKAVHKAVLTIDEK
GTEAAGAMFLEAIPMSIPPEVKFNRPFVFLMIEQNTKSPLFMGKVVNPTQK.

4.-9. (canceled)

10. An isolated polynucleotide molecule comprising a nucleic acid sequence encoding the polypeptide of claim 1.

11. An artificial vector comprising the isolated polynucleotide molecule of claim 10.

12. The artificial vector of claim 11, being an expression vector.

13. A cell comprising
the polypeptide of claim 1.

14. A composition comprising the polypeptide of claim 1,
and an acceptable carrier.

15. A pharmaceutical composition comprising the polypeptide of claim 1, and a pharmaceutically acceptable carrier.

16. The pharmaceutical composition of claim 15, for use in the treatment or prevention of a condition selected from the group consisting of: alpha antitrypsin (AAT) deficiency, an AAT related disease, and inflammation, in a subject in need thereof.

17. A method for treating a subject afflicted with a condition selected from the group consisting of: AAT deficiency, an AAT related disease, and inflammation, comprising administering to said subject a therapeutically effective amount of the polypeptide of claim 1.

18. The method of claim 17, wherein any one of: said polypeptide reduces the abundance of a CD40^{Hi} cell, a CD86^{Hi} cell, or both, in said subject; (ii) said polypeptide reduces the expression level, the secretion level, or both, of a factor selected from the group consisting of: interleukin (IL)-6, and tumor necrosis factor alpha, in said subject; (iii) said polypeptide increases the expression level, the secretion level, or both, of a factor selected from the group consisting of: IL-10, and IL-1Ra, in said subject; and (iv) any combination of (i) to (iii).

19.-20. (canceled)

21. A method for modulating the activity of an immune cell, comprising contacting said immune cell with an effective amount of the polypeptide of claim 1, thereby modulating the activity of the immune cell.

22. The method of claim 21, wherein said immune cell is a neutrophil or a macrophage, optionally wherein said activity comprises: anti-inflammatory activity, protease inhibiting activity, pro-inflammatory activity, or any combination thereof, optionally wherein said immune cell is a cell of a subject, and optionally wherein said subject is afflicted with a condition selected from the group consisting of: AAT deficiency, an AAT related disease, inflammation, and any combination thereof.

23.-25. (canceled)

26. The method of claim 21, wherein said contacting is contacting in vivo or in vitro.

27. A method for producing a polypeptide characterized by having immune cell modulating activity, comprising:
a. providing a cell comprising the artificial vector of claim 11; and
b. culturing said cell of step (a) such that a polypeptide encoded by said artificial vector is expressed,
thereby producing the polypeptide characterized by having immune cell modulating activity.

28. (canceled)

29. The method of claim 27, further comprising a step preceding step (a) comprising introducing or transfecting said cell with said artificial vector.

30. The method of claim 27, wherein said artificial vector is an expression vector.

31. The method of claim 27, further comprising a step proceeding step (b) comprising isolating, extracting, purifying, or any combination thereof, said polypeptide from said cell, from a medium wherein said cell is cultured, or both.

32. A composition comprising the artificial vector of claim 11.


SEQUENCE LISTING (58 sequences)

SEQ ID NO: 1 - protein, 394 aa, Artificial Sequence
  Synthetic
  MISC_FEATURE (22)..(22): X is Thr or Ala
  MISC_FEATURE (68)..(68): X is Thr or Ala
  MISC_FEATURE (74)..(74): X is Asp or Thr
  MISC_FEATURE (202)..(202): X is Asp or His
  misc_feature (243)..(243): Xaa can be any naturally occurring amino acid
  MISC_FEATURE (368)..(368): X is Lys or Arg
EDPQGDAAQKTDTSHHDQDHPXFNKITPNLAEFAFSLYRQLAHQSNSTNIFFSPVSIATAFAMLSLGXKADTHXEILEGLNFNLTEIPEAQIHEGFQELLHTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKXTEEEDFHVDQVTTVKVPMMKRLGMFNIQHCKKLSSWVLLMXYLGNATAIFFLPDEGKLQHLENELTHDIITKFLENEDRRSASLHLPKLSITGTYDLKSILGQLGITKVFSNGADLSGVTEEAPLKLSKAVHKAVLTIDEKGTEAAGAMFLEAIPMSIPPEVKFNXPFVFLMIEQNTKSPLFMGKVVNPTQK

SEQ ID NO: 2 - protein, 394 aa, Artificial Sequence
  Synthetic
EDPQGDAAQKTDTSHHDQDHPAFNKITPNLAEFAFSLYRQLAHQSNSTNIFFSPVSIATAFAMLSLGTKADTHDEILEGLNFNLTEIPEAQIHEGFQELLHTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSILGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 3 - protein, 394 aa, Artificial Sequence
  Synthetic
EDPQGDAAQKTDTSHHDQDHPTFNKITPNLAEFAFSLYRQLAHQSNSTNIFFSPVSIATAFAMLSLGAKADTHDEILEGLNFNLTEIPEAQIHEGFQELLHTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSILGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 4 - protein, 394 aa, Artificial Sequence
  Synthetic
EDPQGDAAQKTDTSHHDQDHPTFNKITPNLAEFAFSLYRQLAHQSNSTNIFFSPVSIATAFAMLSLGTKADTHTEILEGLNFNLTEIPEAQIHEGFQELLHTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSILGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 5 - protein, 394 aa, Artificial Sequence
  Synthetic
EDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLHTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKHTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSILGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 6 - protein, 394 aa, Artificial Sequence
  Synthetic
EDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLHTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMDYLGNATAIFFLPDEGKLQHLENELTHDIITKFLENEDRRSASLHLPKLSITGTYDLKSILGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 7 - protein, 394 aa, Artificial Sequence
  Synthetic
EDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLHTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSILGQLGITKVFSNGADLSGVTEEAPLKLSKAVHKAVLTIDEKGTEAAGAMFLEAIPMSIPPEVKFNRPFVFLMIEQNTKSPLFMGKVVNPTQK

SEQ ID NO: 8 - protein, 394 aa, Artificial Sequence
  Synthetic
EDPQGDAAQKTDTSHHDQDHPAFNKITPNLAEFAFSLYRQLAHQSNSTNIFFSPVSIATAFAMLSLGAKADTHTEILEGLNFNLTEIPEAQIHEGFQELLHTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKHTEEEDFHVDQVTTVKVPMMKRLGMFNIQHCKKLSSWVLLMDYLGNATAIFFLPDEGKLQHLENELTHDIITKFLENEDRRSASLHLPKLSITGTYDLKSILGQLGITKVFSNGADLSGVTEEAPLKLSKAVHKAVLTIDEKGTEAAGAMFLEAIPMSIPPEVKFNRPFVFLMIEQNTKSPLFMGKVVNPTQK

SEQ ID NO: 9 - protein, 394 aa, Artificial Sequence
  Synthetic
EDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLHTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSILGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 10 - DNA, 30 bp, Artificial Sequence
  Synthetic
cacccagccttcaacaagatcacccccaac

SEQ ID NO: 11 - DNA, 29 bp, Artificial Sequence
  Synthetic
gaaggctgggtgatcctgatcatggtggg

SEQ ID NO: 12 - DNA, 24 bp, Artificial Sequence
  Synthetic
ctgggggccaaggctgacactcac

SEQ ID NO: 13 - DNA, 24 bp, Artificial Sequence
  Synthetic
cttggcccccagggagagcattgc

SEQ ID NO: 14 - DNA, 28 bp, Artificial Sequence
  Synthetic
cactcacactgaaatcctggagggcctg

SEQ ID NO: 15 - DNA, 31 bp, Artificial Sequence
  Synthetic
gatttcagtgtgagtgtcagccttggtcccc

SEQ ID NO: 16 - DNA, 31 bp, Artificial Sequence
  Synthetic
gatttcagtgtgagtgtcagccttggccccc

SEQ ID NO: 17 - DNA, 30 bp, Artificial Sequence
  Synthetic
gtcaagcacaccgaggaagaggacttccac

SEQ ID NO: 18 - DNA, 34 bp, Artificial Sequence
  Synthetic
ggtgtgcttgacttcaaagggtctctcccatttg

SEQ ID NO: 19 - DNA, 29 bp, Artificial Sequence
  Synthetic
gctgatggattacctgggcaatgccaccg

SEQ ID NO: 20 - DNA, 30 bp, Artificial Sequence
  Synthetic
ccaggtaatccatcagcagcacccagctgg

SEQ ID NO: 21 - DNA, 48 bp, Artificial Sequence
  Synthetic
caacagaccctttgtcttcttaatgattgaacaaaataccaagtctcc

SEQ ID NO: 22 - DNA, 26 bp, Artificial Sequence
  Synthetic
agggtctgttgaacttgacctcgggg

SEQ ID NO: 23 - DNA, 1345 bp, Artificial Sequence
  Synthetic
acatgtaatcgacaatgccgtcttctgtctcgtggggcatcctcctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctaaaccagccagacagccagctccagctgaccaccggcaatggcctgttcctcagcgagggcctgaagctagtggataagtttttggaggatgttaaaaagttgtaccactcagaagccttcactgtcaacttcggggatcacgaagaggccaagaaacagatcaacgattacgtggagaagggtactcaagggaaaattgtggatttggtcaaggagcttgacagagacacagtttttgctctggtgaattacatcttctttaaaggcaaatgggagagaccttttgaagtcaaggacaccgaggacgaggacttccacgtggaccaggtgaccaccgtgaaggtccctatgatgaagcgtttaggcatgtttaacatccagcactgtaagaagctgtccagctgggtactgctaatgaaatacctgggcaatgccaccgccatcttcttcctacctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaggggactgaagctgctggggccatgtttttagaggccataccaatgtctatccccccagaggtcaagttcaacaaaccctttgtcttcttaatgattgaacaaaataccaagtctcccctcttcatgggaaaagtggtgaatcccacccaaaaataactgcctctcgctcctcaacccctcccctccatccctggccccctccctggatgacattaaagaagggttgagctgga

SEQ ID NO: 24 - DNA, 33 bp, Artificial Sequence
  Synthetic
ggagatgctgcccaggagacagatacatcccac

SEQ ID NO: 25 - DNA, 33 bp, Artificial Sequence
  Synthetic
atcaggatcacccagccttcaacaagatcaccc

SEQ ID NO: 26 - DNA, 32 bp, Artificial Sequence
  Synthtic
caggatcacccaacctgcaacaagatcacccc

SEQ ID NO: 27 - DNA, 33 bp, Artificial Sequence
  Synthetic
gatcacccaaccttccacaagatcacccccaac

SEQ ID NO: 28 - DNA, 33 bp, Artificial Sequence
  Synthetic
accttcaacaagatcgcccccaacctggctgag

SEQ ID NO: 29 - DNA, 32 bp, Artificial Sequence
  Synthetic
ccccaacctggctgatttcgccttcagcctat

SEQ ID NO: 30 - DNA, 33 bp, Artificial Sequence
  Synthetic
gcctataccgccaggtggcacaccagtccaaca

SEQ ID NO: 31 - DNA, 33 bp, Artificial Sequence
  Synthetic
acaccagtccaacaccaccaatatcttcttctc

SEQ ID NO: 32 - DNA, 32 bp, Artificial Sequence
  Synthetic
gctctccctgggggccaaggctgacactcacg

SEQ ID NO: 33 - DNA, 32 bp, Artificial Sequence
  Synthetic
ccctggggaccaagggtgacactcacgatgaa

SEQ ID NO: 34 - DNA, 32 bp, Artificial Sequence
  Synthetic
aaggctgacactcacactgaaatcctggaggg

SEQ ID NO: 35 - DNA, 32 bp, Artificial Sequence
  Synthetic
ctgacactcacgatcaaatcctggagggcctg

SEQ ID NO: 36 - DNA, 33 bp, Artificial Sequence
  Synthetic
acctcacggagattgcggaggctcagatccatg

SEQ ID NO: 37 - DNA, 31 bp, Artificial Sequence
  Synthetic
gcaatggcctgttcatcagcgagggcctgaa

SEQ ID NO: 38 - DNA, 33 bp, Artificial Sequence
  Synthetic
atggcctgttcctcaacgagggcctgaagctag

SEQ ID NO: 39 - DNA, 31 bp, Artificial Sequence
  Synthetic
ctgttcctcagcgagagcctgaagctagtgg

SEQ ID NO: 40 - DNA, 31 bp, Artificial Sequence; identical to SEQ ID NO: 37
  Synthetic

SEQ ID NO: 41 - DNA, 32 bp, Artificial Sequence
  Synthetic
gaccctttgaagtcgaggacaccgaggaagag

SEQ ID NO: 42 - DNA, 32 bp, Artificial Sequence
  Synthetic
cctttgaagtcaagcacaccgaggaagaggac

SEQ ID NO: 43 - DNA, 33 bp, Artificial Sequence
  Synthetic
gaagtcaaggacaccacggaagaggacttccac

SEQ ID NO: 44 - DNA, 33 bp, Artificial Sequence
  Synthetic
gacttccacgtggacgaggtgaccaccgtgaag

SEQ ID NO: 45 - DNA, 33 bp, Artificial Sequence
  Synthetic
cgtttaggcatgtttgacatccagcactgtaag

SEQ ID NO: 46 - DNA, 33 bp, Artificial Sequence
  Synthetic
ggcatgtttaacatccaccactgtaagaagctg

SEQ ID NO: 47 - DNA, 33 bp, Artificial Sequence
  Synthetic
tgggtgctgctgatggactacctgggcaatgcc

SEQ ID NO: 48 - DNA, 32 bp, Artificial Sequence
  Synthetic
ctgctgatgaaatacgtgggcaatgccaccgc

SEQ ID NO: 49 - DNA, 32 bp, Artificial Sequence
  Synthetic
gaaactacagcacctggaaaatgaactcaccc

SEQ ID NO: 50 - DNA, 33 bp, Artificial Sequence
  Synthetic
actcacccacgatatcttaaccaagttcctgga

SEQ ID NO: 51 - DNA, 33 bp, Artificial Sequence
  Synthetic
ccaaactgtccattagtggaacctatgatctga

SEQ ID NO: 52 - DNA, 31 bp, Artificial Sequence
  Synthetic
gctgacctctccgggatcacagaggaggcac

SEQ ID NO: 53 - DNA, 34 bp, Artificial Sequence
  Synthetic
gaagctgctggggccacgtttttagaggccatac

SEQ ID NO: 54 - DNA, 33 bp, Artificial Sequence
  Synthetic
gaggtcaagttcaaccgaccctttgtcttctta

SEQ ID NO: 55 - DNA, 32 bp, Artificial Sequence
  Synthetic
ccctttgtcttcttaatcattgaacaaaatac

SEQ ID NO: 56 - DNA, 33 bp, Artificial Sequence
  Synthetic
ctttgtcttcttaatgtacgaacaaaataccaa

SEQ ID NO: 57 - DNA, 32 bp, Artificial Sequence
  Synthetic
gtcttcttaatgattgaccaaaataccaagtc

SEQ ID NO: 58 - DNA, 34 bp, Artificial Sequence
  Synthetic
aagtctcccctcttcgtgggaaaagtggtgaatc